(19) United States
(12) Patent Application Publication
(10) Pub. No.: US 2026/0165582 A1
(43) Pub. Date: Jun. 18, 2026

(54) IMPLANTABLE CARDIAC DEVICE PATIENT MANAGEMENT

(71) Applicant: Cardiac Pacemakers, Inc., St. Paul, MN (US)

(72) Inventors: Deepa MAHAJAN, North Oaks, MN (US); Sunipa SAHA, Shoreview, MN (US); David L. PERSCHBACHER, St. Paul, MN (US); Keith L. HERRMANN, Minneapolis, MN (US)

(21) Appl. No.: 19/414,646

(22) Filed: Dec. 10, 2025

Related U.S. Application Data
(60) Provisional application No. 63/733,480, filed on Dec. 13, 2024.

(51) Int. Cl. A61B 5/00; A61B 5/01; A61B 5/0205; A61B 5/021; A61B 5/0255; A61B 5/11; A61B 5/145; A61B 5/29; A61B 5/333; A61B 5/361; A61B 5/363; A61B 5/364; A61B 7/02; A61B 7/04

(52) U.S. Cl. CPC A61B 5/0006; A61B 5/01; A61B 5/02055; A61B 5/021; A61B 5/0255; A61B 5/11; A61B 5/14542; A61B 5/29; A61B 5/333; A61B 5/361; A61B 5/363; A61B 5/364; A61B 5/686; A61B 5/746; A61B 7/023; A61B 7/04; A61B 5/742; A61B 5/7475; A61B 2560/0295; A61B 2560/0462; A61B 2562/0204; A61B 2562/0247; A61B 2562/0271

(57) ABSTRACT

Systems and methods to improve resource utilization in ambulatory patient monitoring are disclosed, including receiving a request to record a set of physiologic information of a patient for a patient-triggered episode using one or more sensors of the ambulatory medical device, controlling recording and storage of the set of physiologic information of the patient for the patient-triggered episode, and controlling, at a first time, communication of a first subset of physiologic information to the remote device and storage of a second subset of physiologic information at the ambulatory medical device for selectable separate and subsequent communication to the remote device to improve resource utilization of the ambulatory patient monitoring.

### CROSS REFERENCE TO RELATED APPLICATION

This application claims the benefit of and priority to U.S. Provisional Application No. 63/733,480, filed Dec. 13, 2024, the entire contents of which is hereby incorporated by reference.

### TECHNICAL FIELD

This document relates generally to ambulatory medical devices and more particularly, but not by way of limitation, to systems and methods for improving implantable medical device management and efficiency in ambulatory patient monitoring.

### BACKGROUND

Ambulatory medical devices (AMDs), including implantable, subcutaneous, wearable, insertable, or one or more other medical devices, etc., can monitor, detect, or treat various conditions, including heart failure (HF), arrhythmia, etc. Ambulatory medical devices can include sensors to sense physiologic information from a patient and one or more circuits to detect one or more physiologic events using the sensed physiologic information or transmit sensed physiologic information or detected physiologic events to one or more remote devices. Additionally, ambulatory medical devices can be configured to provide electrical stimulation or one or more other therapies or treatments to the patient, such as to improve cardiac function, etc.

Ambulatory patient monitoring can provide long-term and accurate patient monitoring and early detection of worsening patient condition, including worsening heart failure, arrhythmia, etc. Accurate identification of patients or groups of patients at an elevated risk of future adverse events may control mode or feature selection or resource management of one or more medical devices, control notifications or messages in connected systems to various users associated with a specific patient or group of patients, organize or schedule physician or patient contact or treatment, or prevent or reduce patient hospitalization. Correctly identifying and safely managing patient risk of worsening condition may avoid unnecessary medical interventions, extend the usable life of medical devices, and reduce healthcare costs. In addition, in situations where different operating modes, features, or therapies are available, correctly monitoring, detecting, and identifying patient health status, including improving or worsening patient condition, and modifying one or more medical device functions based thereon, can improve medical device efficiency, such as by reducing unnecessary resource consumption, thereby extending the usable life of the ambulatory medical device.

### SUMMARY

Systems and methods to improve resource utilization in ambulatory patient monitoring are disclosed, including receiving a request to record a set of physiologic information of a patient for a patient-triggered episode using one or more sensors of the ambulatory medical device, controlling recording and storage of the set of physiologic information of the patient for the patient-triggered episode, and controlling, at a first time, communication of a first subset of physiologic information to the remote device and storage of a second subset of physiologic information at the ambulatory medical device for selectable separate and subsequent communication to the remote device to improve resource utilization of the ambulatory patient monitoring.

An example of subject matter (e.g., a medical device system to improve resource utilization in ambulatory patient monitoring) optionally comprises means for receiving a request to record a set of physiologic information of a patient for a patient-triggered episode means for transitioning, in response to receiving the request to record the patient-triggered episode, an ambulatory medical device from a long-term ambulatory monitoring state to a patient-triggered episode state and means for controlling, in the patient-triggered episode state, recording and storage of the set of physiologic information of the patient for the patient-triggered episode and, at a first time, in the patient-triggered episode state, communication of a first subset of physiologic information to a remote device and storage of a second subset of physiologic information at the ambulatory medical device for selectable separate and subsequent communication to the remote device to improve resource utilization of the ambulatory patient monitoring.

In an example, which may be combined with any one or more examples described herein, the subject matter optionally comprises the ambulatory medical device configured to perform long-term ambulatory monitoring of physiologic information of a patient, the ambulatory medical device optionally comprises a communication circuit that is optionally configured to communicate with the remote device, including to receive the request to record the patient-triggered episode and a control circuit that is optionally configured to control operation of the ambulatory medical device, including, in response to receiving the request to record the patient-triggered episode, to transition the ambulatory medical device from the long-term ambulatory monitoring state to the patient-triggered episode state, wherein the control circuit, in the patient-triggered episode state, is optionally configured to control the recording and storage of the set of physiologic information of the patient for the patient-triggered episode and control, at a first time, the communication of the first subset of physiologic information to the remote device and storage of the second subset of physiologic information at the ambulatory medical device for selectable separate and subsequent communication to the remote device to improve resource utilization of the ambulatory patient monitoring.

In an example, which may be combined with any one or more examples described herein, the control circuit, in the patient-triggered episode state, is optionally configured to partition the set of physiologic information of the patient for the patient-triggered episode into the first subset of physiologic information and the second subset of physiologic information, wherein the first subset of physiologic information has a size or duration smaller than a size or duration of the set of physiologic information for the patient-triggered episode, wherein, at the first time, the control circuit is optionally configured to control communication of the first subset of physiologic information to the remote device and storage of the second subset of physiologic information at the ambulatory medical device without communicating the second subset of physiologic information to the remote device at the first time to improve resource utilization of the ambulatory patient monitoring.

In an example, which may be combined with any one or more examples described herein, the ambulatory medical device optionally comprises a cardiac electrical sensor that is optionally configured to sense cardiac electrical information of the patient, wherein, in the long-term ambulatory monitoring state, the cardiac electrical sensor is optionally configured to detect a heart rate or cardiac interval of the patient, wherein, in the patient-triggered episode state, the cardiac electrical sensor is optionally configured to capture a subcutaneous electrocardiogram of the patient having a higher resolution or sampling frequency than the cardiac electrical information detected in the long-term ambulatory monitoring state, wherein the set of physiologic information of the patient for the patient-triggered episode optionally comprises the subcutaneous electrocardiogram.

In an example, which may be combined with any one or more examples described herein, the ambulatory medical device optionally comprises an implantable medical device, wherein the medical device system optionally comprises the remote device and the remote device is optionally configured to receive a request from the patient to record the patient-triggered episode and provide the request to record the patient-triggered episode to the ambulatory medical device receive, in response to the request to record the patient-triggered episode, the first subset of physiologic information from the ambulatory medical device analyze the first subset of physiologic information using one or more algorithms to detect a cardiac rhythm of interest in the first subset of physiologic information and generate, in response to detecting the cardiac rhythm of interest in the first subset of physiologic information, an alert for clinical review of the patient-triggered episode including an indication that additional physiologic information of the patient-triggered episode is available for transmission at the ambulatory medical device.

In an example, which may be combined with any one or more examples described herein, to analyze the first subset of physiologic information to detect the cardiac rhythm of interest includes to detect at least one of atrioventricular block, atrial fibrillation, one or more or a series of premature ventricular contractions, a supraventricular tachycardia, one or more ectopic beats, sinus bradycardia, sinus tachycardia, or junctional rhythms.

In an example, which may be combined with any one or more examples described herein, the remote device is optionally configured to request, in response to detecting the cardiac rhythm of interest in the first subset of physiologic information, the second subset of physiologic information from the ambulatory medical device receive, in response to the request for the second subset of physiologic information, the second subset of physiologic information from the ambulatory medical device and analyze the second subset of physiologic information to detect or confirm the cardiac rhythm of interest in the patient-triggered episode or the second subset of physiologic information, annotate the patient-triggered episode with a label to identify the detected cardiac rhythm of interest, and generate an alert for clinical review of the annotated patient-triggered episode including an indication that additional physiologic information of the patient is available for transmission at the ambulatory medical device.

In an example, which may be combined with any one or more examples described herein, the subject matter optionally comprises a remote server separate from the remote device, wherein the remote server is optionally configured, in response to the remote device detecting the cardiac rhythm of interest in the first subset of physiologic information, to receive the first and second subsets of physiologic information from the ambulatory medical device or the remote device analyze the first and second subsets of physiologic information using a deep learning cardiac algorithm to detect a cardiac arrhythmia in the physiologic information of the patient-triggered episode annotate the patient-triggered episode with a label to identify the detected cardiac rhythm of interest generate a programming change for the ambulatory medical device based on the detected cardiac arrhythmia and generate an alert for clinical review of the annotated patient-triggered episode and the generated programming change.

In an example, which may be combined with any one or more examples described herein, the remote server is optionally configured to prioritize the patient-triggered episode for clinical review based on the detected cardiac rhythm of interest, including to flag critical arrhythmias for urgent clinical review.

In an example, which may be combined with any one or more examples described herein, the communication circuit is optionally configured to receive a request to transmit the second subset of physiologic information to the remote device, wherein the control circuit, in response to receiving the request to transmit the second subset of physiologic information to the remote device, is optionally configured to control, at a second time separate from and subsequent to the first time, communication of the second subset of physiologic information to the remote device or a remote server separate from the remote device.

In an example, which may be combined with any one or more examples described herein, in response to receiving the request to transmit the second subset of physiologic information to the remote device, the control circuit is optionally configured to enable monitoring of additional physiological information of the patient using one or more sensors of the ambulatory medical device and communication of the additional physiologic information to the remote device or the remote server separate from the remote device, the additional physiologic information optionally comprises at least one of blood pressure information using a blood pressure sensor temperature information using a temperature sensor oxygen saturation information using an oxygen saturation sensor photographic information of the patient using an image sensor heart sound information using a heart sound sensor activity information using an activity sensor or impedance information using an impedance sensor.

An example of subject matter (e.g., a method to improve resource utilization in ambulatory patient monitoring) optionally comprises receiving, using a communication circuit of an ambulatory medical device, a request to record a set of physiologic information of a patient for a patient-triggered episode using one or more sensors of the ambulatory medical device transitioning, in response to receiving the request to record the patient-triggered episode, the ambulatory medical device from a long-term ambulatory monitoring state to a patient-triggered episode state controlling, in the patient-triggered episode state, recording and storage of the set of physiologic information of the patient for the patient-triggered episode and controlling, at a first time, in the patient-triggered episode state, communication of a first subset of physiologic information to a remote device and storage of a second subset of physiologic information at the ambulatory medical device for selectable separate and subsequent communication to the remote device to improve resource utilization of the ambulatory patient monitoring.

In an example, which may be combined with any one or more examples described herein, the subject matter optionally comprises partitioning the set of physiologic information of the patient for the patient-triggered episode into the first subset of physiologic information and the second subset of physiologic information, wherein the first subset of physiologic information has a size or duration smaller than a size or duration of the set of physiologic information for the patient-triggered episode, wherein controlling communication of the first subset of physiologic information to the remote device and storage of the second subset of physiologic information at the ambulatory medical device at the first time optionally comprises without communicating the second subset of physiologic information to the remote device at the first time to improve resource utilization of the ambulatory patient monitoring.

In an example, which may be combined with any one or more examples described herein, the subject matter optionally comprises sensing cardiac electrical information of the patient using a cardiac electrical sensor, including in the long-term ambulatory monitoring state, detecting a heart rate or cardiac interval of the patient using the cardiac electrical sensor and in the patient-triggered episode state, capturing a subcutaneous electrocardiogram of the patient using the cardiac electrical sensor, the subcutaneous electrocardiogram having a higher resolution or sampling frequency than the cardiac electrical information detected in the long-term ambulatory monitoring state, wherein the set of physiologic information of the patient for the patient-triggered episode optionally comprises the subcutaneous electrocardiogram.

In an example, which may be combined with any one or more examples described herein, the subject matter optionally comprises, using the remote device receiving a request from the patient to record the patient-triggered episode and provide the request to record the patient-triggered episode to the ambulatory medical device receiving, in response to the request to record the patient-triggered episode, the first subset of physiologic information from the ambulatory medical device analyzing the first subset of physiologic information using one or more algorithms to detect a cardiac rhythm of interest in the first subset of physiologic information and generating, in response to detecting the cardiac rhythm of interest in the first subset of physiologic information, an alert for clinical review of the patient-triggered episode including an indication that additional physiologic information of the patient-triggered episode is available for transmission at the ambulatory medical device.

In an example, a system, method, or apparatus may optionally combine any portion or combination of any portion of any one or more of the examples described herein, may optionally combine any portion or combination of any portion of any one or more of the examples described herein to comprise “means for” performing any portion of any one or more of the functions, operations, or methods of the examples described herein, or at least one “non-transitory machine-readable medium” including instructions that, when performed by a machine, cause the machine to perform any portion of any one or more of the functions or methods of the examples described herein.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example medical device system.

FIGS. 2A-2B illustrate views of an example patient application.

FIG. 3 illustrates an example method.

FIG. 4 illustrates an example medical device system.

FIG. 5 illustrates an example patient management system.

FIG. 6 illustrates example remote patient management systems.

FIG. 7 illustrates a block diagram of an example machine 700 upon which any one or more of the techniques (e.g., methodologies) discussed herein may perform.

### DETAILED DESCRIPTION

Ambulatory medical devices are configured to be implanted in or otherwise positioned on or about patients to provide long-term and accurate monitoring of physiologic information, such as cardiac electrical, heart sound, respiration, impedance, pressure, physical activity, or other physiologic information or one or more other physiologic parameters of the patient, or to provide electrical stimulation or one or more other therapies or treatments to optimize or control one or more body functions of the patient, such as contractions of a heart, etc. Ambulatory medical devices can include implantable or external (e.g., wearable) devices configured to provide long-term and accurate patient monitoring of or in certain examples stimulation to the patient.

Ambulatory medical devices can provide different monitoring, storage, communication, or therapy using different modes with different power and resource requirements and varying sensitivities for different patients. For example, a variety of therapy modalities are available to patients with different power and resource requirements. In certain examples, baseline physiologic information must be established through patient monitoring to determine the optimal medical device, therapy mode, or therapy parameter settings for the patient, as optimal programming often depends on, among other things, accurate detection and determination of different events, conditions, or indications that requires or relies upon such baseline physiologic information. However, resource allocation for physiologic monitoring must be balanced to maintain optimal usable lifespan of the ambulatory medical device. Parameter settings and programming can include, among other things, detection and verification of cardiac capture and determination of relevant capture and stimulation thresholds, selection of pacing or sensing vectors, identification of optimal pacing modes, and resource usage associated with storage of physiologic data or communication or transmission of physiologic data from the ambulatory medical device, etc.

Communication and transmission of physiologic data outside of ambulatory medical devices, such as from an implantable medical device to one or more external devices outside of the body or otherwise, constitute one of the most significant sources of resource consumption, requiring substantial power and processing allocation. Improvements in control of such resources are a significant consideration in managing ambulatory medical devices, particularly in ambulatory medical devices configured to capture patient-triggered episodes (PTE), such as subcutaneous electrocardiogram recordings triggered by a user through a user interface (e.g., through a trigger, command, or symptom received through a patient application operating on a mobile device or a remote patient monitor, etc.).

Existing medical device systems, such as the LUX-Dx™, LUX-Dx II™ or LUX-Dx II+™ insertable cardiac monitor (ICM) or one or more other ambulatory medical devices, are configured to capture and transmit patient-triggered episodes capture, record, analyze, store, and transmit significant amounts of non-actionable data, with up to 64% of transmitted patient-triggered episodes containing no clinically relevant information. Managing patient-triggered episodes and optimizing device operation and resources associated therewith, including managing physiological data of patient-triggered episodes, represent a core technical challenge in implantable and ambulatory medical device technology, and optimization of such, including a reduction in transmitted and analyzed episodes, represents a considerable technical improvement in resource utilization and efficiency in implantable and ambulatory medical device technology.

In an example, patient-triggered episodes can include subcutaneous electrocardiogram recordings having a duration of 5 minutes or greater, such as 7.5 minutes, 10 minutes, etc., and can be analyzed by the ambulatory medical device using one or more algorithms, or separately transmitted, in whole or part, to one or more external devices, such as a remote patient monitor, a medical device programmer, a remote monitoring application such as the myLUX™ Patient App, etc., operating on a smartphone or mobile device of a user, or separately or in combination, a remote server for additional, more intensive analysis requiring additional processing resources not available or efficiently utilized on the ambulatory medical device, or in certain examples in combination with some analysis on the ambulatory medical device (e.g., a first count of valid beats, noise detection, etc.). For example, a deep-learning cardiac algorithm, such as BeatLogic™, etc., performed using a remote server, can improve the detection and analysis of cardiac arrhythmias, particularly atrial fibrillation (AF), utilizing neural network architectures trained on electrocardiogram (ECG) datasets to deliver enhanced sensitivity and specificity in detecting arrhythmias, enabling identification and classification of a broad spectrum of cardiac rhythm abnormalities with a higher sensitivity and specificity than on ambulatory or remote devices. Deep-learning cardiac algorithms, such as BeatLogic™, can utilize neural network architectures trained on electrocardiogram datasets to deliver enhanced sensitivity and specificity in detecting arrhythmias, with demonstrated performance metrics including greater than 99.7% beat detection, greater than 97% sinus sensitivity, greater than 97% VT sensitivity, and greater than 96% AFib sensitivity.

For example, one or more components of the medical device system can detect and classify various atrioventricular (AV) block conditions, including first-degree AV block with normal sinus rhythm, bradycardia, tachycardia, Mobitz Type I and Type II blocks, etc. Additionally, one or more components of the medical device system can identify different presentations of atrial fibrillation, including normal, rapid, and slow variants, along with other complex arrhythmias such as accelerated idioventricular rhythm (AIVR), accelerated junctional rhythm (AJR), junctional escape rhythm (JER), and intraventricular conduction delay (IVCD). The one or more components of the medical device system can detect and classify various ectopic beat patterns, including premature ventricular contractions, supraventricular contractions, and different morphologies of ventricular and supraventricular beats.

In certain examples, patient-triggered episodes can be analyzed, reviewed, and annotated with labels of detected arrhythmias or other events, and prioritized for presentation or display for and review by a clinician on one or more external or remote devices, such as a medical device programmer, etc. In certain examples, one or more alerts can be provided to a clinician or other user to review one or more patient-triggered episodes, etc., such as in response to one or more detected critical events (e.g., ventricular tachycardia (VT), etc.) in the patient-triggered episode, etc. In an example, programming changes for the ambulatory medical device can be recommended based on the one or more detected critical events in the patient-triggered episode, etc.

For example, the present inventors have recognized, among other things, that, in response to patient-triggered episodes or analysis of physiologic information from patient-triggered episodes, additional physiologic monitoring can be enabled on the ambulatory medical device to enhance or improve sensing, recording, transmitting physiologic information or detecting physiologic events. For example, determination of physiologic trends or monitoring of additional physiologic information can be enabled to confirm detected events, increasing resources associated with monitoring the patient for a time period in response to information from the patient-triggered episodes. For example, a patient who is suddenly experiencing symptoms may also demonstrate lower activity or higher fluid retention. Thus, in addition to subcutaneous electrocardiogram, activity monitoring or fluid monitoring (e.g., impedance monitoring, etc.) can be enabled or increased in duration, frequency, or the amount of data recorded in response to the patient-triggered episode to confirm or validate one or more symptoms or other information from the patient-triggered episode. In certain examples, the type of additional monitoring can depend on specific symptoms reported, such as by the patient through the remote monitor or remote monitoring application, etc.

In other examples, supplemental monitoring and data collection by one or more external sensors, such as one or more sensors associated with an external device, such as the smartphone or mobile device of the patient, a wearable medical device (e.g., smartwatch, a fitness tracker, smart earbuds, smart ring, smart glasses, etc.), etc., can be triggered by or in response to the patient-triggered episode. For example, one or more of blood pressure, temperature, oxygen saturation, or photographs, videos, or voice recordings of the user (e.g., of the eyes, facial expression, etc.) or requests or instructions therefor can be triggered in response to the patient-triggered episode, received, and analyzed to supplement or prioritize the patient-triggered episode, to confirm or verify one or more detected events or symptoms associated with the patient-triggered episode, etc. In certain examples, transmission, analysis, or upload of the detected information can be limited until confirmation of the patient-triggered episode by one or more algorithms, detections, or agreement by information in the supplemental monitoring and data collection, or subsequent request by a clinician or algorithm after review or analysis of the initial limited action.

For example, if premature ventricular contractions (PVCs) are detected at elevated levels in the patient-triggered episode, such the number of detected premature ventricular contractions in the patient-triggered episode exceeds a threshold (e.g., 5% of the number of beats in the patient-triggered episodes, etc.), a dedicated premature ventricular contraction detection algorithm can be enabled in the ambulatory medical device, such as for a monitoring period after exceeding the threshold, to record and transmit additional diagnostic data for confirmation or verification. Such targeted additional monitoring can ensure comprehensive capture of potentially significant ventricular arrhythmias. In an example, the monitoring period can include one or more periods commensurate with the patient-triggered episode. In other examples, the monitoring period can include one or more longer time periods, such as 2-4 weeks, etc.

In an example, the medical device system, comprising one or more components of a patient management system, can implement intelligent programming based on one or more detected arrhythmic patterns or determined changes in patient status, patient response metrics, etc., such as described in the commonly assigned Horan et al. U.S. Patent Application Ser. No. 63/655,782 titled “REMOTE CRT PARAMETER CHANGE RESPONSE EVALUATION AND PROGRAMMING,” or in the commonly assigned Rajan et al. U.S. Patent Application Ser. No. 63/604,724 titled “ARRYTHMIA DETECTION REPROGRAMMING RECOMMENDATION ALGORITHM,” each of which are hereby incorporated by reference in their entireties, including their disclosures of generating reprogramming recommendations or remotely reprogramming an implantable medical device. For example, the patient management system can generate recommendations for adjusting programming parameters or automatically modifying ambulatory medical device settings to optimize future detection of these or other arrhythmias by the ambulatory medical device or one or more other components of the patient management system. These automated monitoring and programming capabilities can combine to create an adaptive system to optimize or improve arrhythmia detection or detection of one or more other events to improve diagnostic accuracy and enhance patient care management, while optimizing and improving resource allocation in medical device systems and associated components.

In other examples, in response to a patient-triggered episode, a first subset of information shorter than the entire patient-triggered episode in time or smaller than the patient-triggered episode in size (e.g., representative, partial, reduced, or summary information, for example, downsampled to a lower frequency from a higher-frequency signal, etc.) can be transmitted from the ambulatory medical device for analysis to one or more components of an external system, such as a remote patient monitor, a medical device programmer, a remote monitoring application operating on a smartphone or mobile device of a user, or in certain examples, separately or in combination with a remote server. In an example, the external system can analyze the first subset of information and, if including clinically relevant information, such as one or more events, rhythms, conditions, or metrics exceeding a threshold or satisfying a condition (e.g., indicative of patient worsening, abnormal rhythm, etc.), one or more components of the external system can instruct the ambulatory medical device to transmit the additional information from the patient-triggered episode (e.g., a second subset of information, the remainder of the patient-triggered episode after the first subset, an original higher-frequency signal, etc.).

In certain examples, if the first subset of information includes clinically relevant information, such as determined by one or more algorithms or processes of an external system or remote server, the external system or remote server can mark the first subset as part of a larger patient-triggered episode and cause the first subset of information to be provided to a clinician for review on a display or an alert of such first subset to be provided to the clinician including the mark that additional information is available for transmission, analysis, and review. In certain examples, the additional information can be transmitted in response to one or more of clinician confirmation of the first subset including clinically relevant information or a received clinician request for additional information. In other examples, the first subset of information can be sufficient, and the remainder of the information of the patient-triggered episode can remain on ambulatory medical device, such as for a period of time or otherwise marked for deletion, to be overwritten, etc.

Partitioning the patient-triggered episode into separate subsets additionally improves the speed at which the patient-triggered episode is analyzed by the external or remote device. Patient-triggered episodes are typically not communicated outside of the ambulatory medical device until after the duration of the patient-triggered episode has completed, and then the entire episode is transmitted if satisfying some condition of the ambulatory medical device. Communicating the first subset of the patient-triggered episode before recording the entire patient-triggered episode, which can have a duration of 5, 7.5, or even 10-mminutes or greater, can significantly reduce latency associated with analysis of potentially critical cardiac rhythms, such as VT, etc.

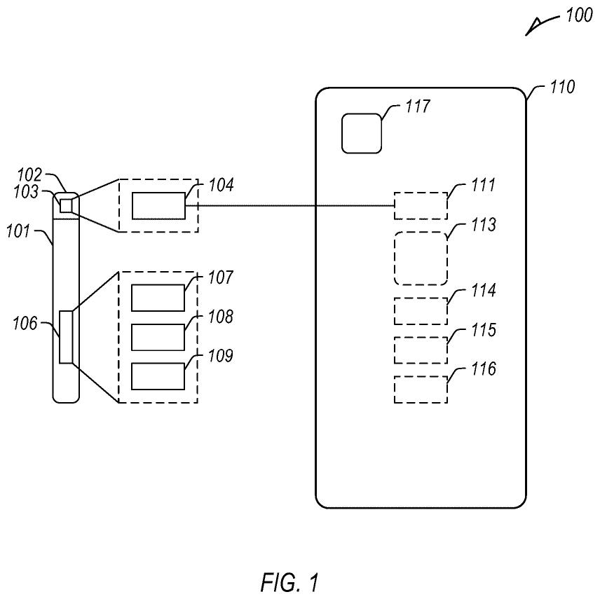

FIG. 1 illustrates an example medical device system 100 including an implantable medical device 101, such as an insertable cardiac monitor (ICM) or other implantable medical device, and a remote device 110, such as a cellular phone, a medical device programmer, or one or more other portable or other electronic device. In various examples, the remote device 110 can be one or more component of the external system 505 described in FIG. 5 (e.g., the external device 506, the remote device 508, etc.).

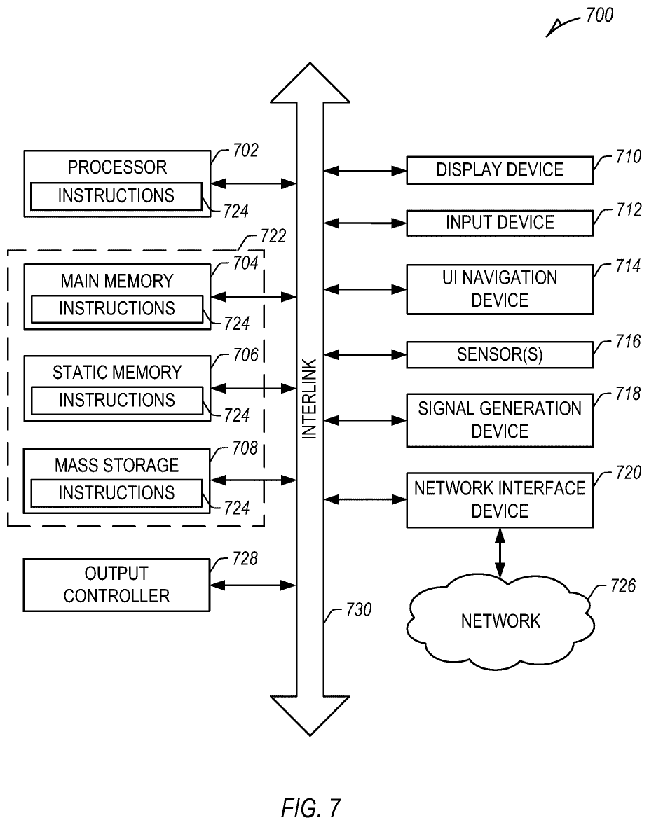

The implantable medical device 101 can comprise a housing (or “can”) composed of a biocompatible material, such as titanium, etc., and can include a header 102 composed of a biocompatible or implantable grade polymer. In certain examples, one or both of the housing and the header 102 can include one or more electrodes. The header can include one or more circuits 103, such as one or both of a communication circuit 104 of the implantable medical device 101 (or one or more components thereof, such as an antenna, etc.). The housing of the implantable medical device 101 includes additional circuits 106, such as a control circuit 107, a memory circuit 108, a power source 109 (e.g., a battery), etc. Additionally, the implantable medical device 101 can include a data bus to enable communication between different components of the implantable medical device (e.g., similar to the interlink 730 illustrated in FIG. 7). In certain examples, the data bus can include different portions, such as a first portion between one or more sensors of the implantable medical device and the control circuit 107 or the memory circuit 108, and a second portion between the communication circuit 104 and the control circuit 107 or the memory circuit 108, etc.

The remote device 110 includes a remote communication circuit 111 configured to communicate with the communication circuit 104 of the implantable medical device 101, a magnet 113 (e.g., to wake up or initiate communication with the implantable medical device 101), and one or more other circuit components, such as a control circuit 114, a memory circuit 115, a power source 116 (e.g., a battery), etc. In certain examples, the magnet 113 can be a component of a protective case of the remote device 110.

In an example, the remote device 110 can include or be configured to execute a patient application 117 to communicate with the implantable medical device 101. In certain examples, the remote device 110 can perform initial processing of physiologic information received from the implantable medical device 101, such as to confirm an initial cardiac rhythm of interest or to separately screen for excess noise or normal sinus rhythm in a first or other portions of physiologic information of a patient-triggered episode. In certain examples, the remote device 110 can be configured to communicate with one or more other devices, including a remote server for additional processing, etc.

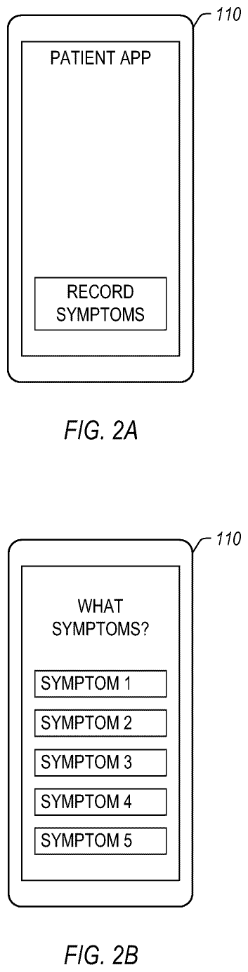

FIGS. 2A-2B illustrate views of a patient application operating on a mobile device 110. The patient application (e.g., myLUX™) can enable patients to activate and communicate with an implanted medical device, such as an insertable cardiac monitor (e.g., LUX-Dx II™ or LUX-Dx II+™ insertable cardiac monitor, etc.). The patient application can be configured to enable communication of patient information from the implantable medical device to a remote device or remote server (e.g., a LATITUDE™ server, etc.). The patient application can provide user-friendly features designed to help empower patients and increase compliance, including monitoring and connection status displays, step-by-step reconnection instructions, and a symptom tracker that allows patients to record symptoms and activities in real-time. For example, the patient application, in FIG. 2A, includes a “RECORD SYMPTOMS” button selectable by the patient that can provide a request to the implantable medical device to record a patient-triggered episode.

In certain examples, the patient application can include one or more selectable symptoms, such as “SYMPTOM 1” through “SYMPTOM 5” illustrated in FIG. 2B. Depending on the provided symptoms, or in certain examples, the determined cardiac rhythm of interest, the mobile device of the patient can be configured to record certain patient information, including a voice, photo, or video recording describing the symptoms or in proximity to occurrence. In other examples, one or more sensors of the mobile device can be used to record other physiologic information of the patient, such as using one or more connected wearable devices or other sensors of or coupled to the mobile device, etc.

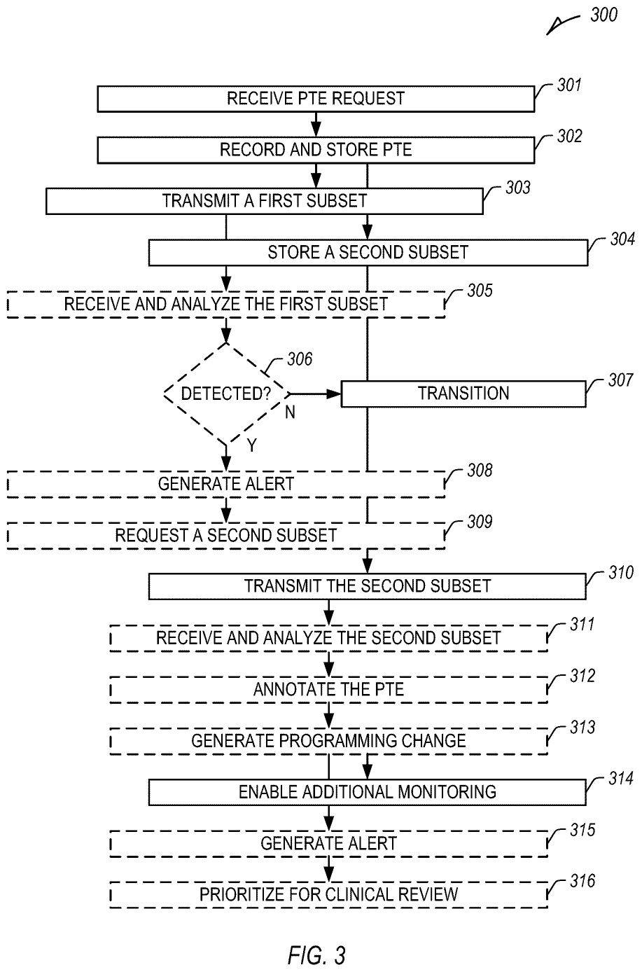

FIG. 3 illustrates an example method 300 to improve resource utilization in ambulatory patient monitoring, such as by an ambulatory medical device configured to perform long-term ambulatory monitoring of physiologic information of a patient using one or more sensors of the ambulatory medical device.

In an example, an external or remote device separate from the ambulatory medical device, but in certain examples a component of a medical device system, can receive a request from a patient to record a patient-triggered episode, such as through one or more user interfaces available to the patient, including a patient application operating on a mobile device or a remote patient monitor, etc. In other examples, instead of a specific request to record a patient-triggered episode, the request can include the receipt or reporting of one or more symptoms by the patient. In an example, the external or remote device can be configured to provide the request to record the patient-triggered episode to the ambulatory medical device.

At step 301, the request to record a patient-triggered episode can be received, such as using a communication circuit of an ambulatory medical device, and performed, such as using one or more sensors of the ambulatory medical device, in certain examples controlled by a control circuit of the ambulatory medical device. In an example, the ambulatory medical device can be configured to transition, in response to receiving the request to record the patient-triggered episode, from a long-term ambulatory monitoring state or one or more ambulatory monitoring states to a patient-triggered episode state, such as using a control circuit of the ambulatory medical device.

At step 302, a set of physiologic information of the patient for the patient-triggered episode can be recorded and stored in the patient-triggered episode state, such as in response to receiving the request to record the patient-triggered episode. The control circuit can be configured to control operation of the ambulatory medical device, including to control recording and storage of physiologic information for the patient-triggered episode having a higher resolution or sampling frequency or additional sensing, sampling, or storage than in the long-term ambulatory monitoring state.

For example, cardiac electrical information can be detected in both the long-term ambulatory monitoring state and the patient-triggered episode state. In an example, a heart rate or cardiac interval of the patient can be detected using the cardiac electrical sensor or photoplethysmography (PPG) sensor in the long-term ambulatory monitoring state, whereas in the patient-triggered episode state, a subcutaneous electrocardiogram of the patient can be detected and recorded with a higher resolution or sampling frequency compared to the cardiac electrical information detected in the long-term ambulatory monitoring state. The enhanced detail and accuracy of the subcutaneous electrocardiogram during the patient-triggered episode can facilitate more precise detection and analysis of cardiac events.

At step 303, a first subset of the set of physiologic information can be transmitted, such as using the communication circuit, to one or more external or remote devices. In an example, the set of physiologic information can be partitioned into first and second, and in certain examples, one or more other subsets, such as using the control circuit or one or more other circuits or components of the ambulatory medical device.

In an example, the first subset can be a first portion of the set of physiologic information, including a first portion of the set recorded and stored in time or as it occurs, and as such, can be transmitted (or transmission can begin), in certain examples, before the remainder of the set of physiologic information of the set of physiologic information of the patient-triggered episode has been recorded and stored by the ambulatory medical device. In such example, not only is transmitting the first portion before transmitting the second portion a reduction of ambulatory monitoring resources, such as to omit transmitting at least a portion if not all of the 64% of patient-triggered episodes that do not include actionable cardiac rhythms, but transmitting the first portion also reduces the time at which initial processing of the set of physiologic information is performed, reducing the latency of subsequent review, alerts, or changes to one or more parameter settings of the ambulatory medical device.

In other examples, the first subset can be transmitted, such as after recording and storing the set of physiologic information of the patient-triggered episode. In an example, the first subset may not be a first portion, but may be a most interesting portion as determined by one or more algorithms operating on the ambulatory medical device, such as determined by the control circuit of the ambulatory medical device, for example, using a first level or processing resources, etc. In other examples, the first subset can include summary information of the set of physiologic information of the patient-triggered episode, including for example, descriptions or statistics of rhythms, a representation of the set of physiologic information, such as reduced in resolution, frequency, size, or one or more other characteristics, etc., such that additional information of the set of physiologic information exists at the ambulatory medical device for selectable and optional transmission at a later time, such as by request of the external or remote device, etc. In certain examples, the set of physiologic information of the patient-triggered episode can include information from one sensor, or in other examples, information from a plurality of sensors or a plurality of information from one or more sensors, where the first subset includes a portion of the recorded information but not all of the recorded information from the patient-triggered episode.

At step 304, a second subset of the set of physiologic information can be stored, such as controlled by the control circuit using one or more memory or storage circuits of the ambulatory medical device. The second subset can be stored at the ambulatory medical device for selectable separate and subsequent communication to the external or remote device to improve resource utilization of the ambulatory patient monitoring. For example, if the first subset is analyzed and no cardiac rhythm of interest is found, the second subset may not be communicated outside of the ambulator medical device, improving resource utilization of the ambulatory medical device.

At step 305, the first subset of physiologic information from the ambulatory medical device can be received, such as at the external or remote device, for example, in response to the request to record the patient-triggered episode.

At step 306, the first subset of physiologic information can be analyzed using one or more algorithms one the external or remote device to detect a cardiac rhythm of interest in the first subset of physiologic information. In an example, the first subset of physiologic information can be analyzed to detect, in the first subset of physiologic information, evidence of one or more of the following cardiac rhythms: atrioventricular block; atrial fibrillation; ventricular tachycardia; one or more or a series of premature ventricular contractions (e.g., a ventricular run); a ventricular pause greater than a pause threshold (e.g., 3 seconds, etc.); a supraventricular tachyarrhythmia (SVTA) event greater than an SVTA threshold (e.g., greater than 6 seconds, etc.) or having a low confidence (e.g., requiring additional confirmation, etc.); a relative count of ectopic beats above an ectopic beat threshold (e.g., greater than 5% of valid beats in a window, such as a subset or patient-triggered episode, etc.); occurrence of sinus bradycardia and sinus tachycardia in a subset or patient-triggered episode both appearing greater than a threshold (e.g., 6 seconds or greater, etc.); evidence of junctional rhythms originating from the atrioventricular junction, such as characterized by a lack of normal atrial activity or inverted P waves, or occurring when the sinoatrial node fails to function properly or electrical propagation is blocked; etc. In an example, such analysis can be performed by the external device, such as a mobile device of the patient, etc., such as using a patient application operating on the mobile device accessing one or more external or remote resources, by a remote device, such as remote server resource using one or more deep-learning algorithms, such as BeatLogic™, etc., or combinations thereof.

At step 307, if no cardiac rhythm of interest is detected at step 306, a notification of such, or after confirmation of receipt of the first subset and no subsequent request for a second subset after a threshold amount of time, in certain examples, the ambulatory medical device can be configured to transition from the patient-triggered episode state to the long-term ambulatory monitoring or one or more other ambulatory or long-term monitoring states.

At step 308, if a cardiac rhythm of interest is detected at step 306, an alert can be generated, such as an alert for clinical review of the patient-triggered episode including an indication that additional physiologic information of the patient-triggered episode is available for transmission at the ambulatory medical device.

At step 309, the second subset of physiologic information can be requested by the external or remote device. At step 310, the second subset of physiologic information can be communicated, such as in response to the request generated at step 309, by the ambulatory medical device to the external or remote device, such as using the communication circuit or one or more other circuits or components. In an example, the ambulatory medical device can be configured to transmit the second subset or a remainder of the patient-triggered episode from the ambulatory medical device at a second time separate from and subsequent to the first time, to the external or remote device, a remote server separate from the remote device, etc.

At step 311, the second subset or one or more additional subsets or additional physiologic information from the ambulatory medical device can be received, such as at the external or remote device or one or more components thereof, etc., in response to the request generated at step 309, and in certain examples, analyzed by the external or remote device or one or more components thereof, etc., to detect or confirm the cardiac rhythm of interest in the patient-triggered episode or the second subset of physiologic information.

At step 312, the patient-triggered episode can be annotated, such as using one or more algorithms operating on the external or remote device or one or more components thereof, etc., such as BeatLogic™ or one or more other algorithms, to identify and label rhythms and features in the electrocardiogram of the patient-triggered episode, etc.

At step 313, one or more programming changes can be generated, such as by the external or remote device or one or more components thereof, etc., for example, using the intelligent programming described and incorporated herein. In certain examples, the programming changes can be proposed to a clinician for review, revision, or acceptance, and then provided to an ambulatory medical device for implementation, or, if previously authorized or within a range of previously authorized changes, automatically provided to the ambulatory medical device for implementation. In certain examples, programming changes can include implementation of additional patient monitoring, such as by one or more additional sensors, etc.

At step 314, additional monitoring can be enabled at the ambulatory medical device or one or more other devices, such as the external device, etc. In an example, the additional monitoring can be enabled in response to one or more received programming changes. In other examples, receiving the request to transmit the second subset can automatically enable additional monitoring, such that analysis of the second subset can include an indicator that additional physiologic information is available at the ambulatory medical device to provide additional context of the patient-triggered episode, to confirm a detected cardiac rhythm of interest, etc.

In an example, the additional monitoring can include at least one of: blood pressure information using a blood pressure sensor of the ambulatory medical device or one or more other devices; temperature information using a temperature sensor of the ambulatory medical device or one or more other devices; oxygen saturation information using an oxygen saturation sensor of the ambulatory medical device or one or more other devices, such as a wearable device, etc.; photographic information of the patient using an image sensor, such as of an external device, a mobile device of the patient, etc.; heart sound information using a heart sound sensor of the ambulatory medical device or one or more other devices; activity information using an activity sensor of the ambulatory medical device or one or more other devices; impedance information using an impedance sensor of the ambulatory medical device or one or more other devices; etc.

At step 315, an alert can be generated and provided to a clinician or one or more other processes, such as to enable one or more additional changes, to automatically adjust a patient follow-up schedule, etc. In an example, the alert can include an alert for clinical review of the annotated patient-triggered episode including an indication that additional physiologic information of the patient is available at the ambulatory medical device for transmission to the external or remote device or one or more other devices, etc.

At step 316, if multiple patient-triggered episodes are detected, such as from this patient or one or more other patients, a priority for each patient-triggered episode can be determined, and the multiple patient-triggered episodes can be prioritized for clinical review, such as by a clinician or process, etc. For example, critical rhythms, such as ventricular tachycardia, etc., can be flagged and prioritized, including alerts, for urgent clinical review by a clinician.

Although described herein as an external or remote device, one or more processes described herein can be performed by or using a remote server separate from the external or remote device. For example, in response to the remote device detecting the cardiac rhythm of interest in the first subset of physiologic information, the remote server can be configured to receive the first and second subsets of physiologic information from the ambulatory medical device or the remote device; analyze the first and second subsets of physiologic information using a deep learning cardiac algorithm to detect a cardiac arrhythmia in the physiologic information of the patient-triggered episode; annotate the patient-triggered episode with a label to identify the detected cardiac rhythm of interest; generate a programming change for the ambulatory medical device based on the detected cardiac arrhythmia; and generate an alert for clinical review of the annotated patient-triggered episode and the generated programming change. In other examples, the remote device can include the remote server configured to receive information from the ambulatory medical device through the external device, etc.

Although illustrated as a series of steps, in certain examples, one or more steps can be omitted or be optional. In other examples, different combinations or permutations of these or other steps or examples can be combined to form other methods or processes, which is also applicable to other examples discussed herein.

### Medical Device System

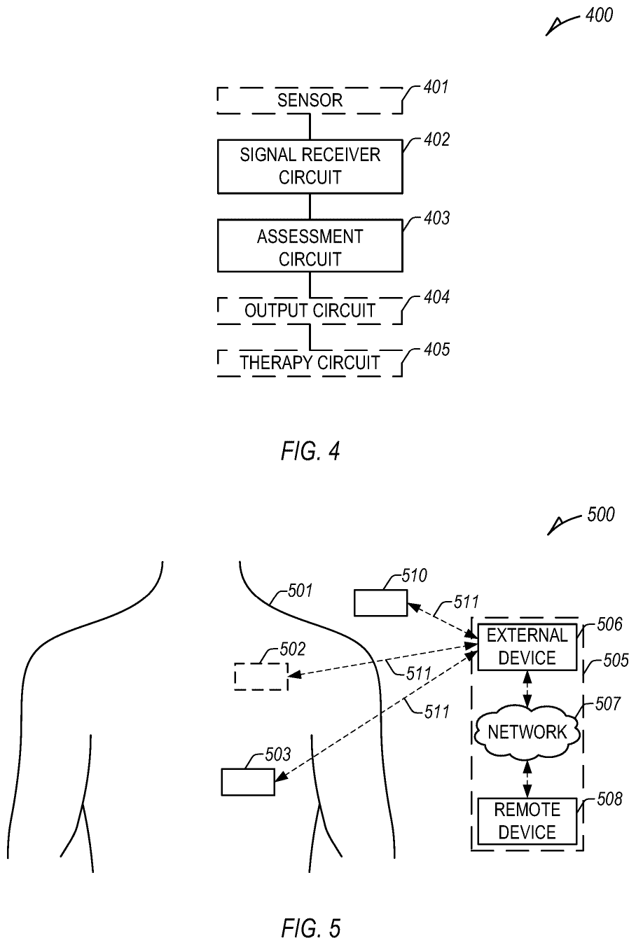

FIG. 4 illustrates an example system 400 (e.g., a medical device system). In an example, one or more aspects of the system 400 can be a component of, or communicatively coupled to, a medical device, such as an implantable medical device (IMD), an insertable cardiac monitor (ICM), an ambulatory medical device (AMD), etc. The system 400 can be configured to monitor, detect, or treat various physiologic conditions of the body, such as cardiac conditions associated with a reduced ability of a heart to sufficiently deliver blood to a body, including heart failure, arrhythmias, dyssynchrony, etc., or one or more other physiologic conditions and, in certain examples, can be configured to provide electrical stimulation or one or more other therapies or treatments to the patient.

The system 400 can include a single medical device or a plurality of medical devices implanted in a body of a patient or otherwise positioned on or about the patient to monitor patient physiologic information of the patient using information from one or more sensors, such as a sensor 401. In an example, the sensor 401 can include one or more of: a respiration sensor configured to receive respiration information (e.g., a respiratory rate, a respiration volume (tidal volume), etc.); an acceleration sensor (e.g., an accelerometer, a microphone, etc.) configured to receive cardiac acceleration information (e.g., cardiac vibration information, pressure waveform information, heart sound information, endocardial acceleration information, acceleration information, activity information, posture information, etc.); an impedance sensor (e.g., an intrathoracic impedance sensor, a transthoracic impedance sensor, a thoracic impedance sensor, etc.) configured to receive impedance information, a cardiac sensor configured to receive cardiac electrical information; an activity sensor configured to receive information about a physical motion (e.g., activity, steps, etc.); a posture sensor configured to receive posture or position information; a pressure sensor configured to receive pressure information; a plethysmograph sensor (e.g., a photoplethysmography sensor, etc.); a chemical sensor (e.g., an electrolyte sensor, a pH sensor, an anion gap sensor, a potassium sensor, a creatinine sensor, etc.); a temperature sensor; a skin elasticity sensor, or one or more other sensors configured to receive physiologic information of the patient.

The example system 400 can include a signal receiver circuit 402 and an assessment circuit 403. The signal receiver circuit 402 can be configured to receive physiologic information of a patient (or group of patients) from the sensor 401. The assessment circuit 403 can be configured to receive information from the signal receiver circuit 402, and to determine one or more parameters (e.g., physiologic parameters, stratifiers, etc.) or existing or changed patient conditions (e.g., indications of patient dehydration, respiratory condition, cardiac condition (e.g., heart failure, arrhythmia), sleep disordered breathing, etc.) using the received physiologic information, such as described herein. Physiologic information can include, among other things, one or more of: electrical information of the patient, such as cardiac electrical information (e.g., heart rate, heart rate variability, etc.), impedance information, temperature information, and in certain examples, respiration information (e.g., a respiratory rate, a respiration volume (tidal volume), etc.); mechanical information of the patient, such as cardiac acceleration information (e.g., cardiac vibration information, pressure waveform information, heart sound information, endocardial acceleration information, acceleration information, activity information, posture information, etc.), physical activity information (e.g., activity, steps, etc.), posture or position information, pressure information, plethysmograph information, and in certain examples, respiration information; chemical information; or other physiologic information of the patient. In an example, the signal receiver circuit 402 can include the sensor 401. In other examples, the signal receiver circuit can be coupled to or a component of the assessment circuit 403.

In certain examples, the assessment circuit 403 can aggregate information from multiple sensors or devices, detect various events using information from each sensor or device separately or in combination, update a detection status for one or more patients based on the information, and transmit a message or an alert to one or more remote devices that a detection for the one or more patients has been made or that information has been stored or transmitted, such that one or more additional processes or systems can use the stored or transmitted detection or information for one or more other review or processes.

In certain examples, such as to detect an improved or worsening patient condition, some initial assessment is often required to establish a baseline level or condition from one or more sensors or physiologic information. Subsequent detection of a deviation from the baseline level or condition can be used to determine the improved or worsening patient condition. However, in other examples, the amount of variation or change (e.g., relative or absolute change) in physiologic information over different time periods can used to determine a risk of an adverse medical event, or to predict or stratify the risk of the patient experiencing an adverse medical event (e.g., a heart failure event) in a period following the detected change, in combination with or separate from any baseline level or condition.

Changes in different physiologic information can be aggregated and weighted based on one or more patient-specific stratifiers and, in certain examples, compared to one or more thresholds, for example, having a clinical sensitivity and specificity across a target population with respect to a specific condition (e.g., heart failure), etc., and one or more specific time periods, such as daily values, short term averages (e.g., daily values aggregated over a number of days), long term averages (e.g., daily values aggregated over a number of short term periods or a greater number of days (sometimes different (e.g., non-overlapping) days than used for the short term average)), etc.

In certain examples, the assessment circuit 403 can aggregate information from multiple sensors or devices, detect various events using information from each sensor or device separately or in combination, update a detection status for one or more patients based on the information, and transmit a message or an alert to one or more remote devices that a detection for the one or more patients has been made or that information has been stored or transmitted, such that one or more additional processes or systems can use the stored or transmitted detection or information for one or more other review or processes.

In certain examples, such as to detect an improved or worsening patient condition, some initial assessment is often required to establish a baseline level or condition from one or more sensors or physiologic information. Subsequent detection of a deviation from the baseline level or condition can be used to determine the improved or worsening patient condition. However, in other examples, the amount of variation or change (e.g., relative or absolute change) in physiologic information over different time periods can used to determine a risk of an adverse medical event, or to predict or stratify the risk of the patient experiencing an adverse medical event (e.g., a heart failure event) in a period following the detected change, in combination with or separate from any baseline level or condition.

Changes in different physiologic information can be aggregated and weighted based on one or more patient-specific stratifiers and, in certain examples, compared to one or more thresholds, for example, having a clinical sensitivity and specificity across a target population with respect to a specific condition (e.g., heart failure), etc., and one or more specific time periods, such as daily values, short term averages (e.g., daily values aggregated over a number of days), long term averages (e.g., daily values aggregated over a number of short term periods or a greater number of days (sometimes different (e.g., non-overlapping) days than used for the short term average)), etc.

The system 400 can include an output circuit 404 configured to provide an output to a user, or to cause an output to be provided to a user, such as through an output, a display, or one or more other user interface, the output including a score, a trend, an alert, or other indication. In other examples, the output circuit 404 can be configured to provide an output to another circuit, machine, or process, such as a therapy circuit 405 (e.g., a cardiac resynchronization therapy (CRT) circuit, a chemical therapy circuit, a stimulation circuit, etc.), etc., to control, adjust, or cease a therapy of a medical device, a drug delivery system, etc., or otherwise alter one or more processes or functions of one or more other aspects of a medical device system, such as one or more CRT parameters, drug delivery, dosage determinations or recommendations, etc. In an example, the therapy circuit 405 can include one or more of a stimulation control circuit, a cardiac stimulation circuit, a neural stimulation circuit, a dosage determination or control circuit, etc. In other examples, the therapy circuit 405 can be controlled by the assessment circuit 403, or one or more other circuits, etc. In certain examples, the assessment circuit 403 can include the output circuit 404 or can be configured to determine the output to be provided by the output circuit 404, while the output circuit 404 can provide the signals that cause the user interface to provide the output to the user based on the output determined by the assessment circuit 403.

### Efficiency and Mode Transitions

Ambulatory medical devices powered by rechargeable or non-rechargeable batteries, responsible for sensing physiologic signals and physiologic information of the patient, and in certain examples making determinations using such information, have to make certain tradeoffs between device battery life, or in the instance of implantable medical devices with non-rechargeable batteries, between device replacement periods often including surgical procedures, and device sensing, storage, processing, and communication characteristics, such as sensing resolution, sampling frequency, sampling periods, the number of active sensors, the amount of stored information, processing characteristics, or communication of physiologic information outside of the device.

Ambulatory medical devices can include higher-power modes and lower-power modes. In certain examples, the low-power mode can include a low resource mode, characterized as requiring less power, processing time, memory, or communication time or bandwidth (e.g., transferring less data, etc.) than a corresponding high-power mode. The high-power mode can include a relatively higher resource mode, characterized as requiring more power, processing time, memory, or communication time or bandwidth than the corresponding low-power mode.

A technological problem in the art with respect to such devices exists that not all information can be stored, not all sensors can be active in a high-power or high-resolution mode, not all algorithms can be active, and not all sensed or processed information can be communicated outside of the device at all times without detrimentally impacting the lifespan of the devices. Technological solutions to such problems are often improvements in physical sensors, or alternatively in sensing and processing physiologic information in a way that improves device efficiency, extending the lifespan of the device, or to perform new determinations using existing sensors or information in a way that was not previously known, increasing the capabilities of an existing device without adding additional hardware to the device, or requiring additional sensors or hardware to be implanted in the patient. Efficiency improvements in one area can enable additional operation in another, improving the technical capabilities of existing devices having real-world constraints.

For example, physiologic information, such as indicative of a potential adverse physiologic event, can be used to transition from a low-power mode to a high-power mode. However, by the time physiologic information detected in the low-power mode indicates a possible event, valuable information has been lost, unable to be recorded in the high-power mode.

Another technological problem exists in that false or inaccurate determinations that trigger a high-power mode unnecessarily unduly limit the usable life of certain ambulatory medical devices. For numerous reasons, it is advantageous to accurately detect and determine physiologic events, and to avoid unnecessary transitions from the low-power mode to the high-power mode to improve use of medical device resources.

In an example, a change in modes can enable higher resolution sampling or an increase in the sampling frequency or number or types of sensors used to sense physiologic information leading up to and including a potential event. Different physiologic information is often sensed using non-overlapping time periods of the same sensor, in certain examples, at different sampling frequencies and power costs.

In certain examples, a high-power mode can be in contrast to a low-power mode, and can include one or more of: enabling one or more additional sensors, transitioning from a low-power sensor or set of sensors to a higher-power sensor or set of sensors, triggering additional sensing from one or more additional sensors or medical devices, increasing a sensing frequency or a sensing or storage resolution, increasing an amount of data to be collected, communicated (e.g., from a first medical device to a second medical device, etc.), or stored, triggering storage of currently available information from a loop recorder in long-term storage or increasing the storage capacity or time period of a loop recorder, or otherwise altering device behavior to capture additional or higher-resolution physiologic information or perform more processing, etc.

Additionally, or alternatively, event storage can be triggered. Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected event (e.g., a heart failure event, an arrhythmia episode, etc.) can be stored, such as to increase the specificity of detection. In an example, multiple loop recorder windows (e.g., 2-minute windows) can be stored sequentially. In systems without early detection, to record this information, a loop recorder with a longer time period would be required at substantial additional cost (e.g., power, processing resources, component cost, etc.).

For example, ambulatory medical devices frequently contain one or more accelerometer sensors and corresponding processing circuits to determine and monitor patient acceleration information, such as, among other things, cardiac vibration information associated with blood flow or movement in the heart or patient vasculature (e.g., heart sounds, cardiac wall motion, etc.), patient physical activity or position information (e.g., patient posture, activity, etc.), respiration information (e.g., respiratory rate (RR), tidal volume (TV), rapid shallow breathing index (RSBI), respiration phase, breathing sounds, etc.), etc. In one example, heart sounds and patient activity can be detected using non-overlapping time periods of the same, single- or multi-axis accelerometer, at different sampling frequencies and power costs.

In an example, a transition to a high-power mode can include using the accelerometer to detect heart sounds throughout the high-power mode, or at a larger percentage of the high-power mode than during a corresponding low-power mode, etc. In other examples, waveforms for medical events can be recorded, stored in long-term memory, and transferred to a remote device for clinician review. In certain examples, only a notification that an event has been stored is transferred, or summary information about the event. In response, the full event can be requested for subsequent transmission and review. However, even in the situation where the event is stored and not transmitted, resources for storing and processing the event are still by the medical device.

Another technological problem exists in that suboptimal programming of device parameters and parameter settings can negatively impact functionality of ambulatory medical devices. Accordingly, identifying suboptimal programming by clinicians and other caregivers and generating and providing alerts or notifications of such identified suboptimal programming, or reprogramming recommendations, and in certain examples, reprogramming ambulatory medical devices directly, can improve the functionality of existing ambulatory medical devices without requiring other improvements to the hardware of devices providing therapy or the sensors themselves.

### Implant and Follow-Up

When receiving a new medical device, patients may need to try several sets of parameter settings to receive sufficient or optimal therapy. In addition, in-clinic follow-up appointments are currently required as patient condition can change or response to existing devices or therapy can change over time, requiring additional changes to parameter settings that at one time were sufficient or optimal. In typical operation, a medical device, such as a cardiac resynchronization therapy device, is first programmed at a time of implant to a first operating mode, such as with respect to a first set of parameter settings, and then adjusted during scheduled in-clinic follow-up procedures with a clinician. Initial follow-up after implant (e.g., post-implant) or programming changes is generally a first time period, such as 4-6 weeks to allow patient recovery from the implant procedure and determination of baseline measurements for the patient from which to base future operation and monitoring, as well as to compare patient condition after implant or programming changes to the patient condition pre-implant or previous to programming changes. Subsequent follow-up after the initial follow-up can be less frequent, occurring, for example, every 3-6 months (e.g., at 6 months post-implant, at 12-months post-implant, etc.), or more or less as needed (e.g., between 1 and 12 months, etc.) depending on programming changes or changes in patient health status or condition (e.g., a patient response metric). However, traditional follow-up appointments are in-person in a clinical setting and require travel for the patient, often at a substantial burden. In addition, programming changes may require additional follow-up, such as a new initial follow-up appointment and observation time, substantially increasing resources associated with programming the medical device and reducing the usable lifespan of the medical device during which the device is using limited resources to provide sufficient or optimal therapy.

In other examples, follow-up schedules can be determined or prioritized based on information from the ambulatory medical device, and moreover, changes can be made remotely, without in-person follow-up appointments, increasing the usable lifespan of the limited resources of the ambulatory medical device.

For implantable medical devices, once implanted and in certain examples after a recovery period, the implantable medical device can begin in a first mode, such as a cardiac resynchronization therapy mode, a monitoring mode, or one or more other therapy modes having different parameter settings, depending on the patient or clinician. In certain examples, a stimulation circuit can generate and provide one or more stimulation signals in one or more stimulation modes, and the assessment circuit can be configured to control the stimulation circuit, such as to adjust one or more parameters or transition between different therapy modes, etc.

### Physiologic Parameters

In certain examples, physiologic information of a patient can be sensed using one or more sensors located within, on, or proximate to the patient, such as a cardiac sensor, a heart sound sensor, or one or more other sensors described herein. For example, cardiac electrical information of the patient can be sensed using a cardiac sensor. In other examples, cardiac acceleration information of the patient can be sensed using a heart sound sensor. The cardiac sensor and the heart sound sensor can be components of one or more (e.g., the same or different) medical devices (e.g., an implantable medical device, an ambulatory medical device, etc.). Timing metrics between different features (e.g., first and second cardiac features, etc.) can be determined, such as by a processing circuit of the cardiac sensor or one or more other medical devices or medical device components, etc. In certain examples, the timing metric can include an interval or metric between first and second cardiac features of a first cardiac interval of the patient (e.g., a duration of a cardiac cycle or interval, a QRS width, etc.) or between first and second cardiac features of respective successive first and second cardiac intervals of the patient. In an example, the first and second cardiac features include equivalent detected features in successive first and second cardiac intervals, such as successive R waves (e.g., an R-R interval, etc.) or one or more other features of the cardiac electrical signal, etc.

Heart sounds are recurring mechanical signals associated with cardiac vibrations or accelerations from blood flow through the heart or other cardiac movements with each cardiac cycle or interval and can be separated and classified according to activity associated with such vibrations, accelerations, movements, pressure waves, or blood flow. Heart sounds include four major features: the first through the fourth heart sounds (S1 through S4, respectively). The first heart sound (S1) is the vibrational sound made by the heart during closure of the atrioventricular (AV) valves, the mitral valve and the tricuspid valve, and the opening of the aortic valve at the beginning of systole, or ventricular contraction. The second heart sound (S2) is the vibrational sound made by the heart during closure of the aortic and pulmonary valves at the beginning of diastole, or ventricular relaxation. The third and fourth heart sounds (S3, S4) are related to filling pressures of the left ventricle (LV) during diastole. An abrupt halt of early diastolic filling can cause the third heart sound (S3). Vibrations due to atrial kick can cause the fourth heart sound (S4). Valve closures and blood movement and pressure changes in the heart can cause accelerations, vibrations, or movement of the cardiac walls that can be detected using an accelerometer or a microphone, providing an output referred to herein as cardiac acceleration information.

In an example, heart sound signal portions, or values of respective heart sound signals for a cardiac interval, can be detected as amplitudes occurring with respect to one or more cardiac electrical features or one or more energy values with respect to a window of the heart sound signal, often determined with respect to one or more cardiac electrical features. For example, the value and timing of an S1 signal can be detected using an amplitude or energy of the heart sound signal occurring at or about the R wave of the cardiac interval, and the value and timing of an S2 signal can be detected using an amplitude or energy of the heart sound signal occurring at or about the closure of the aortic and pulmonary valves, marking the transition from systole to diastole. S3 and S4 signal portions can be determined, such as by a processing circuit of the heart sound sensor or one or more other medical devices or medical device components, etc. In certain examples, the S3 signal portion can include a value or energy of the heart sound signal occurring in an S3 window of the cardiac interval, and the S4 signal portion can include a value or energy of the heart sound signal occurring in an S4 window of the cardiac interval. The S3 window occurs after S2 (e.g., 100 ms-200 ms after S2, 150 ms-200 ms after S2, etc.) and lasts for an S3 interval (e.g., 100 ms, 200 ms, etc.). The S4 window occurs shortly before the R wave or S1 (ending before or at the R wave or S1) and lasts for an S4 interval (e.g., 50 ms, 100 ms, 200 ms, etc.). The S3 or S4 windows and intervals can be determined as a set time period in the cardiac interval with respect to one or more other cardiac electrical or mechanical features, such as forward from one or more of the R wave, the T wave, or one or more features of a heart sound waveform or backwards from a subsequent R wave or a detected S1 of a subsequent cardiac interval. In certain examples, the length of the S3 or S4 intervals can depend on one or more factors, such as heart rate or patient characteristics, etc.

In an example, a heart sound parameter can include information of or about multiple of the same heart sound parameter or different combinations of heart sound parameters over one or more cardiac cycles or a specified time period (e.g., 1 minute, 1 hour, 1 day, 1 week, etc.). For example, a heart sound parameter can include a composite first heart sound (S1) parameter representative of a plurality of S1 parameters, for example, over a certain time period (e.g., a number of cardiac cycles, a representative time period, etc.), or one or more other heart sounds (e.g., a second heart sound (S2), a third heart sound (S3), a fourth heart sound (S4), etc.), etc.

In an example, the heart sound parameter can include an ensemble average of a particular heart sound over a heart sound waveform, such as that disclosed in the commonly assigned Siejko et al. U.S. Pat. No. 7,115,096 entitled “THIRD HEART SOUND ACTIVITY INDEX FOR HEART FAILURE MONITORING,” or in the commonly assigned Patangay et al. U.S. Pat. No. 7,853,327 entitled “HEART SOUND TRACKING SYSTEM AND METHOD,” each of which are hereby incorporated by reference in their entireties, including their disclosures of ensemble averaging an acoustic signal and determining a particular heart sound of a heart sound waveform. In other examples, the signal receiver circuit can receive the at least one heart sound parameter or composite parameter, such as from a heart sound sensor or a heart sound sensor circuit.

In an example, cardiac electrical information of the patient can be received, such as using a signal receiver circuit of a medical device, from a cardiac sensor (e.g., one or more electrodes, etc.) or cardiac sensor circuit (e.g., including one or more amplifier or filter circuits, etc.). In an example, the received cardiac electrical information can include the timing metric between the first and second cardiac features of the patient. In an example, cardiac acceleration information of the patient can be received, such as using the same or different signal receiver circuit of the medical device, from a heart sound sensor (e.g., an accelerometer, etc.) or heart sound sensor circuit (e.g., including one or more amplifier or filter circuits, etc.). In certain examples, additional physiologic information can be received, such as one or more of heart rate information, activity information of the patient, or posture information of the patient, from one or more other sensor or sensor circuits.

Respiration information can include, among other things, a respiratory rate (RR) of the patient, a tidal volume (TV) of the patient, a rapid shallow breathing index (RSBI) of the patient, or other respiratory information of the patient. The respiratory rate is a measure of a breathing rate of the patient, generally measured in breaths per minute. The tidal volume is an aggregate measure of respiration changes, such as detected using measured changes in thoracic impedance, etc. The RSBI is a measure (e.g., a ratio) of respiratory frequency relative to (e.g., divided by) tidal volume of the patient. The nHR is a measure of heart rate (HR) of the patient at night, either in relation to sensing patient sleep or using a preset or selectable time of day corresponding to patient sleep. In certain examples, respiration information of the patient can be determined using changes in impedance information and accordingly can be considered electrical information, but different than cardiac electrical information. In other examples, respiration information of the patient can be determined using changes in activity or acceleration information and accordingly can be considered mechanical information.

Physiologic metrics, as described herein, or measures or indications of physiologic information, can include one or more different measures of rate, amplitude, energy, etc., of different physiologic information over one or more time periods, such as representative daily values, etc. For example, heart sound metrics can be determined for each heart sound (e.g., the first heart sound (S1) through the fourth heart sound (S4), etc.) and can include an indication of an amplitude or energy of a specific heart sound for a specific cardiac cycle, or a representation of a number of cardiac cycles of the patient over a specific time period. Daily metrics can be determined representative of an average daily value for the patient, either corresponding to a waking time or a 24-hour period, etc. Respiration metrics can include, among other things, a mean or median respiratory rate, binned values of rates, and a representative value of specific rate bins, etc. Heart rate metrics can include an average nighttime heart rate, a minimum nighttime heart rate, heart rate at rest, etc.

The activity information can include an activity measurement of the patient, such as detected using an accelerometer, a posture sensor, a step counter, or one or more other activity sensors associated with an ambulatory medical device. Activity may be used to gate other physiologic measurements such as heart rate or respiratory rate so that the change in these metrics with increased patient activity may be used to infer patient cardiovascular and metabolic status including measurement of oxygen consumption. The impedance information can include, among other things, thoracic impedance information of the patient, such as a measure of impedance across a thorax of the patient from one or more electrodes associated with the ambulatory medical device (e.g., one or more leads of an implantable medical device proximate a heart of the patient and a housing of the implantable medical device implanted subcutaneously at a thoracic location of the patient, one or more external leads on a body of the patient, etc.). In other examples, the impedance information can include one or more other impedance measurements associated with the thorax of the patient, or otherwise indicative of patient thoracic impedance.

The temperature information can include an internal patient temperature at an ambulatory medical device, such as implanted in the thorax of the patient, or one or more other temperature measurements made at a specific location on the patient, etc. The temperature information can be detected using a temperature sensor, such as one or more circuits or electronic components having an electrical characteristic that changes with temperature. The temperature sensor can include a sensing element located on, at, or within the ambulatory medical device configured to determine a temperature indicative of patient temperature at the location of the ambulatory medical device.

In contrast to and separate from the electrical or mechanical information discussed above, the chemical information can include information about one or more chemical properties of blood, interstitial space (e.g., the space between cells, such as including interstitial fluid), or other tissue (e.g., muscle tissue, fat tissue, organ tissue, etc.) of the patient, such as information indicative of or including one or more of a glucose level, pH level, dissolved gas level (e.g. oxygen, carbon dioxide, carbon monoxide, etc.), electrolyte level (e.g., sodium, potassium, calcium, etc.), organic compound level (e.g., lactate, cholesterol, hemoglobin, creatinine, etc.), or biologic compound level (e.g., enzymes, antibodies, receptors, etc.), etc. The chemical information may be measured by one or more of an electrical sensor, mechanical sensor, electrochemical sensor, biosensor (e.g., enzyme biosensor, etc.), ion-selective electrode sensor, optical sensor, etc. In an example, the chemical information may include potassium information (e.g., one or more of interstitial potassium information, serum potassium information, etc.), creatinine information (e.g., one or more of interstitial creatinine information, serum creatinine information, etc.), or combinations thereof.

In certain examples, interstitial chemical information, such as one or more chemical levels in an interstitial space (e.g., a space between one or more of connective tissue, muscle fibers, nervous tissue, etc.) or of interstitial fluid, etc., can be indicative of serum chemical information. For example, potassium may move between cells or tissue and interstitial fluid (e.g., a change in interstitial potassium level may be followed by or reflective of a change in serum potassium level or vice versa), such that chemical information on serum potassium can include interstitial potassium. In certain examples, one of interstitial or serum chemical information can lead or lag the other, such that a change in one can indicate a worsening patient condition is detectable before the other. In one example, interstitial potassium information can lead serum potassium information as an indicator of electrolyte imbalance.

### Alert States

In certain examples, an alert state (e.g., an in-alert state, an out-of-alert state, a priority alert state, etc.) of the patient can be adjusted or determined using information of the patient, such as to increase a sensitivity or specificity of alert state determination, reduce false positive alert state determinations, alert state transitions or adjustments, or otherwise reduce storage or transmission of physiologic information associated or transitions associated with false positive alert state determinations, and power and processing resources associated with the same. In an example, the alert state can be determined using a comparison of a value of the health index (e.g., a numerical value, etc.) to one or more fixed or adaptable alert thresholds (e.g., based at least in part on one or more relative factors, such as measurements from the patient over the past 30 days, etc.). In an example, the alert state can be provided to a user interface for display to a user or to a control circuit to control or adjust a process or function of the system. In an example, the alert state can include one or more of an indication, recommendation, or instruction to perform one or more actions (e.g., administer or provide a drug or class of drug, adjust or optimize a guideline-directed medical therapy (GDMT), etc.). For, example, a GDMT may advise administration of a quantity of a drug or a rate of increase in a dosage, etc. In an example, determination of an in-alert or priority alert state can trigger an indication or instruction to administer or provide a specific class of diuretic or to deviate from GDMT (e.g., increase GDMT above a standard recommendation, hold GDMT at a standard recommendation, hold GDMT at a current level, decrease GDMT below a standard recommendation, increase a dosage or rate of increase of a drug, reduce a dosage or rate of decrease of a drug, etc.).

In certain examples, the techniques described above or herein can be used in various combinations or permutations. For example, combinations or permutations of techniques described above or herein can be selected based upon patient history, patient treatment (e.g., in-patient care, out-patient care, etc.), clinician input, etc.

As used herein, high and low (or high, medium, and low, etc.) can be relative or categorical terms, in certain examples with respect to clinical or population values, patient-specific values (e.g., a representative value, such as a current value, with respect to a short- or long-term range of values, etc.), or combinations thereof. For example, a high value can include a value in an upper percentage (e.g., at or above an upper quartile, etc.) of values experienced by the patient over respective time periods, such as one or more of a short-term range (e.g., having a period between 1 week and 3 months, such as 1 month, etc.), a long term range (e.g., having a period greater than the short-term range, such as greater than 1 month, greater than 3 months, the last 6 months, or longer, etc.). A low value can include a value in a lower percentage (e.g., at or below a mean or median, below the upper quartile, etc.). A medium value can, in certain examples, include a value between the upper and lower quartiles or within a threshold percentage of a mean or median, etc. In other examples, values can be determined with respect to clinical or population values, in certain examples, further respective to matching patient demographics (e.g., age, sex, comorbidities, etc.) or type of medical device (e.g., CRT-D device, ICD device, etc.), etc.

In an example, determinations described herein can be used to change device behavior, trigger additional sensing, data processing, storage, or transmission, or otherwise alter one or more modes, processes, or functions of medical devices associated with such determinations. For example, determinations can require data over a substantial time period (e.g., multiple days, weeks, a month or more, etc.). Such determinations can be initially determined by the device at yearly or semi-yearly (e.g., every 6 months, every 3 months, etc.) by default, or triggered by worsening patient health status or upon instruction from a clinician or caregiver, etc. In a first example, an assessment circuit can determine one or more indications quarterly, consuming a default amount of device resources. If the quarterly determination exceeds one or more of a patient-specific or population threshold, the assessment circuit can alter device functionality to increase the frequency of making such determinations, increasing the use of device resources, in certain examples reducing device lifespan, but providing additional monitoring and determinations. In other examples, if a determination exceeds one or more thresholds, additional sensing can be triggered, such as enabling additional sensors, or sensing enabled sensors with a higher resolution or sampling frequency, storing more information, and communicating more information outside of the device, such as to an external programmer, or increasing the frequency of communication outside of the device, increasing the use of device resources, in certain examples reducing device lifespan, but providing additional monitoring and determinations.

In certain examples, determinations described herein can include one or more determined risk curves illustrating determined risks at different time periods into the future, such as a determined risk of mortality (e.g., cardiovascular death), a determined risk of heart failure hospitalization, etc. Information about the determined risks or the determined risk curves or portions of the determined risk curves themselves can be provided to a user, such as to a patient, clinician, caregiver, etc., or can be used to make one or more device changes, such as described herein (e.g., therapies, treatments, device settings, etc.), or trigger one or more other processes or notifications, etc.

### Patient Indications

Indications of patient condition (e.g., improved or worsening patient condition, etc.) can include single-feature determinations based on a single feature or measure of a single type of physiologic information, or separately a composite determination based on a combination of physiologic information, such as two or more separate features of physiologic measures. In addition, indications of patient condition can be device-based, such as determined using physiologic information detected from the patient using the one or more ambulatory medical devices without input of clinical information about the patient separate from that detected or sensed physiologic information. In other examples, indications of patient condition can be a combination of device-based and clinical-based information of the patient, such as clinician diagnosis or determination of risk, patient history, patient age, comorbidities, prior hospitalization, type of implanted device, etc. In certain examples, separate determinations can be made for different combinations of clinical information.

One example of a composite indication is the HeartLogic™ index, a HeartLogic™ in-alert time, or one or more other composite measurements or measures thereof. The HeartLogic™ index is a composite indication of patient condition determined using different combinations or weightings of physiologic information, including two or more of S1 heart sounds, S3 heart sounds, thoracic impedance, activity information, respiration information, and nighttime heart rate (nHR). The HeartLogic™ index can be indicative of a heart failure status, a risk a heart failure event (e.g., within in a given time period), or a worsening of the heart failure status or risk of heart failure event in the patient over time. The HeartLogic™ in-alert time is a measure of time that the HeartLogic™ index is above an alert threshold.

In certain examples, the different combinations or weightings of physiologic information used to determine the HeartLogic™ index can be adjusted or determined based on a risk stratifier. In certain examples, the risk stratifier can be determined as a different combination of physiologic information, including one or more of S3, respiratory rate, and time active (e.g., an amount of time at a specific activity level above a mean activity level of the patient or a specific threshold, etc.). For example, if the risk stratifier is low, or below a first threshold, the HeartLogic™ index can be determined using a first combination of physiologic information. If the risk stratifier is high, or above a second threshold, the HeartLogic™ index can be determined using a second combination of physiologic information, such as additional information than included in the first combination (e.g., the first combination and the second combination, etc.). If the risk stratifier is between the first and second thresholds, the HeartLogic™ index can be determined using the first combination and one or more metrics or components of the second combination, or using the first combination and the second combination, but with the second combination having less weight than if the risk stratifier is above the second threshold (e.g., using less of the second combination than the first combination).

In an example, the HeartLogic™ index and in-alert time can include worsening heart failure or physiologic event detection, including risk indication or stratification, such as that disclosed in the commonly assigned An et al. U.S. Pat. No. 9,968,266 entitled “RISK STRATIFICATION BASED HEART FAILURE DETECTION ALGORITHM,” or in the commonly assigned An et al. U.S. Pat. No. 9,622,664 entitled “METHODS AND APPARATUS FOR DETECTING HEART FAILURE DECOMPENSATION EVENT AND STRATIFYING THE RISK OF THE SAME,” or in the commonly assigned Thakur et al. U.S. Pat. No. 10,660,577 entitled “SYSTEMS AND METHODS FOR DETECTING WORSENING HEART FAILURE,” or in the commonly assigned An et al. U.S. Patent Application No. 2014/0031643 entitled “HEART FAILURE PATIENT STRATIFICATION,” or in the commonly assigned Thakur et al. U.S. Pat. No. 10,085,696 entitled “DETECTION OF WORSENING HEART FAILURE EVENTS USING HEART SOUNDS,” each of which are hereby incorporated by reference in their entireties, including their disclosures of heart failure and worsening heart failure detection, heart failure risk indication detection, and stratification of the same, etc.

### Patient Management System

FIG. 5 illustrates an example patient management system 500 and portions of an environment in which the patient management system 500 may operate. The patient management system 500 can perform a range of activities, including remote patient monitoring and diagnosis of a disease condition, programming of ambulatory medical devices, and control of one or more therapies. Such activities can be performed proximal to a patient 501, such as in a patient home or office, through a centralized server, such as in a hospital, clinic, or physician office, or through a remote workstation, such as a secure wireless mobile computing device.

The patient management system 500 can include one or more medical devices, an external system 505, and a communication link 511 providing for communication between the one or more ambulatory medical devices and the external system 505. The one or more medical devices can include an ambulatory medical device, such as an implantable medical device 502, a wearable medical device 503, or one or more other implantable, leadless, subcutaneous, external, wearable, or medical devices configured to monitor, sense, or detect information from, determine physiologic information about, or provide one or more therapies to treat various conditions of the patient 501, such as one or more cardiac or non-cardiac conditions (e.g., dehydration, sleep disordered breathing, etc.).

In an example, the implantable medical device 502 can include one or more cardiac rhythm management (CRM) devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 501. In another example, the implantable medical device 502 can include a monitor implanted, for example, subcutaneously in the chest of patient 501, the implantable medical device 502 including a housing containing circuitry and, in certain examples, one or more sensors, such as a temperature sensor, etc.

Cardiac rhythm management devices are generally configured to receive cardiac electrical information from, and in certain examples, provide electrical stimulation to, one or more electrodes located within, on, or proximate to the heart, such as coupled to one or more leads and located in one or more chambers of the heart, within the vasculature of the heart near one or more chambers, or otherwise attached to or in contact with or proximate to the heart. Cardiac rhythm management devices can include, among others, pacemakers, implantable cardioverter defibrillators (ICD), subcutaneous implantable cardioverter defibrillators, cardiac resynchronization therapy defibrillators (CRT-Ds), insertable cardiac monitors, leadless cardiac pacemakers (LCPs), or wearable or remote monitoring systems.

Cardiac resynchronization therapy (CRT) refers generally to stimulation therapy generated and provided to one or more chambers of the heart (e.g., frequently two or more of the right ventricle (RV), the left ventricle (e.g., commonly through the cardiac vasculature), or the right atrium (RA), etc.) to improve cardiac function, such as to improve coordination of contractions between different chambers of the heart (e.g., the right ventricle and the left ventricle, the right atrium and the right ventricle, etc.) or to otherwise improve cardiac output or efficiency. Cardiac resynchronization therapy can include biventricular pacing (e.g., both right and left ventricular pacing), single-chamber pacing (e.g., right ventricle pacing, left ventricle pacing, etc.), sensing or pacing in one or more other chambers or combinations of chambers (e.g., right atria, etc.), as well as multi-site pacing (MSP) (e.g., applying one or more stimulation signals to multiple (e.g., two or more) electrodes in or proximate to a chamber (e.g., commonly the left ventricle, but also in certain examples the right ventricle, the right atrium, or combinations thereof) for a single cardiac cycle), and in certain examples, HIS-bundle pacing, septal pacing, etc. The timing of stimulation signals in the cardiac cycle or with respect to one or more cardiac events often varies depending on a number of factors, including placement of the lead or electrodes, propagation of the stimulation signals through the tissue, and stimulation parameters, such as stimulation amplitude, type, timing, etc.

Accordingly, cardiac rhythm management devices can include aspects located subcutaneously, though proximate the distal skin of the patient, as well as aspects, such as leads or electrodes, located near one or more organs of the patient. Separate from, or in addition to, the one or more electrodes or other sensors of the leads, the cardiac rhythm management device can include one or more electrodes or other sensors (e.g., a pressure sensor, an accelerometer, a gyroscope, a microphone, etc.) powered by a power source in the cardiac rhythm management device. The one or more electrodes or other sensors of the leads, the cardiac rhythm management device, or a combination thereof, can be configured to detect physiologic information from the patient, or provide one or more therapies or stimulation to the patient.

Implantable devices can additionally or separately include leadless cardiac pacemakers, small (e.g., smaller than traditional implantable cardiac rhythm management devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart without traditional lead or implantable cardiac rhythm management device complications (e.g., required incision and pocket, complications associated with lead placement, breakage, or migration, etc.). In certain examples, leadless cardiac pacemakers can have more limited power and processing capabilities than a traditional cardiac rhythm management device; however, multiple leadless cardiac pacemaker devices can be implanted in or about the heart to detect physiologic information from, or provide one or more therapies or stimulation to, one or more chambers of the heart. The multiple leadless cardiac pacemaker devices can communicate between themselves, or one or more other implanted or external devices.

The implantable medical device 502 can include a signal receiver circuit or an assessment circuit configured to detect or determine specific physiologic information of the patient 501, or to determine one or more conditions or provide information or an alert to a user, such as the patient 501 (e.g., a patient), a clinician, or one or more other caregivers or processes, such as described herein. The implantable medical device 502 can alternatively or additionally be configured as a therapeutic device configured to treat one or more medical conditions of the patient 501. The therapy can be delivered to the patient 501 via the lead system and associated electrodes or using one or more other delivery mechanisms. The therapy can include delivery of one or more drugs to the patient 501, such as using the implantable medical device 502 or one or more of the other ambulatory medical devices, etc. In some examples, therapy can include cardiac resynchronization therapy for rectifying dyssynchrony and improving cardiac function in heart failure patients. In other examples, the implantable medical device 502 can include a drug delivery system, such as a drug infusion pump to deliver drugs to the patient for managing arrhythmias or complications from arrhythmias, hypertension, hypotension, or one or more other physiologic conditions. In other examples, the implantable medical device 502 can include one or more electrodes configured to stimulate the nervous system of the patient or to provide stimulation to the muscles of the patient airway, etc.

The wearable medical device 503 can include one or more wearable or external medical sensors or devices (e.g., automatic external defibrillators (AEDs), Holter monitors, patch-based devices, smart watches, smart accessories, wrist- or finger-worn medical devices, such as a finger-based photoplethysmography sensor, etc.). The wearable medical device 503 can include a signal receiver circuit or an assessment circuit configured to detect or determine specific physiologic information of the patient 501, or to determine one or more conditions or provide information or an alert to a user, such as the patient 501 (e.g., a patient), a clinician, or one or more other caregivers or processes, such as described herein.

The external system 505 can include a dedicated hardware/software system, such as a programmer, a remote server-based patient management system, or alternatively a system defined predominantly by software running on a standard personal computer. The external system 505 can manage the patient 501 through the implantable medical device 502 or one or more other ambulatory medical devices connected to the external system 505 via a communication link 511. In other examples, the implantable medical device 502 can be connected to the wearable medical device 503, or the wearable medical device 503 can be connected to the external system 505, via the communication link 511. This can include, for example, programming or reprogramming the implantable medical device 502 with different parameter settings to perform one or more of acquiring physiologic data, performing at least one self-diagnostic test (such as for a device operational status), analyzing the physiologic data, or optionally delivering or adjusting a therapy for the patient 501. Additionally, the external system 505 can send information to, or receive information from, the implantable medical device 502 or the wearable medical device 503 via the communication link 511. Examples of the information can include real-time or stored physiologic data from the patient 501, diagnostic data, such as detection of patient hydration status, hospitalizations, responses to therapies delivered to the patient 501, or device operational status of the implantable medical device 502 or the wearable medical device 503 (e.g., battery status, lead impedance, etc.). The communication link 511 can be an inductive telemetry link, a capacitive telemetry link, or a radio frequency (RF) telemetry link, such as a wireless telemetry based on, for example, Bluetooth® or IEEE 802.11 wireless fidelity “Wi-Fi” interfacing standards. Other configurations and combinations of patient data source interfacing are possible.

The external system 505 can include an external device 506 in proximity of the one or more ambulatory medical devices, and a remote device 508 in a location relatively distant from the one or more ambulatory medical devices, in communication with the external device 506 via a communication network 507. Examples of the external device 506 can include a medical device programmer. The external device 506 or the remote device 508 can be configured to evaluate collected device or patient information and provide alert notifications, among other possible functions. In an example, one or more of the external system 505, the external device 506, or the remote device 508 can include a signal receiver circuit or an assessment circuit configured to receive or determine specific physiologic information of the patient 501, such as from the implantable medical device 502 or the wearable medical device 503, or to determine one or more conditions or provide information or an alert to a user, such as the patient 501 (e.g., a patient), a clinician, or one or more other caregivers or processes, such as described herein.

In an example, the remote device 508 can include a centralized server acting as a central hub for collected data storage and analysis from a number of different sources. Combinations of information from the multiple sources can be used to make determinations and update individual patient health status or to adjust one or more alerts or determinations for one or more other patients. The server can be configured as a uni-, multi-, or distributed computing and processing system. The remote device 508 can receive data from multiple patients. The data can be collected by the one or more ambulatory medical devices, among other data acquisition sensors or devices associated with the patient 501. The server can include a memory device to store the data in a patient database. The server can include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications can include a Web page update, phone or pager call, E-mail, SMS, text, or “Instant” message, as well as a message to the patient and a simultaneous direct notification to emergency services and to the clinician. Other alert notifications are possible. The server can include an alert prioritizer circuit configured to prioritize the alert notifications. For example, an alert of a detected medical event can be prioritized using a similarity metric between the physiologic data associated with the detected medical event to physiologic data associated with the historical alerts.

In an example, similar to the alert notifications discussed above, the external system 505 or one or more components thereof (e.g., the external device 506, the remote device 508, an assessment circuit, etc.) can be configured to schedule one or more follow-up appointments or adjust a schedule of one or more follow-up appointments for the patient such as in response to one or more alert notifications or other determinations, per a request of a clinician, etc.

The remote device 508 may additionally include one or more locally configured clients or remote clients securely connected over the communication network 507 to the server. Examples of the clients can include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 508, including the server and the interconnected clients, may also execute a follow-up scheme by sending follow-up requests to the one or more ambulatory medical devices, or by sending a message or other communication to the patient 501 (e.g., the patient), clinician or authorized third party as a compliance notification.

The communication network 507 can provide wired or wireless interconnectivity. In an example, the communication network 507 can be based on the Transmission Control Protocol/Internet Protocol (TCP/IP) network communication specification, although other types or combinations of networking implementations are possible. Similarly, other network topologies and arrangements are possible.

One or more of the external device 506 or the remote device 508 can output the detected medical events to a system user, such as the patient or a clinician, or to a process including, for example, an instance of a computer program executable in a microprocessor. In an example, the process can include an automated generation of a programming recommendation for an ambulatory medical device to optimize or improve patient condition or otherwise provide a desired clinical outcome. In an example, the external device 506 or the remote device 508 can include a respective display unit for displaying the physiologic or functional signals, or alerts, alarms, emergency calls, or other forms of warnings to signal the detection of one or more conditions. In some examples, the external system 505 can include a signal receiver circuit and an assessment circuit, such as an external data processor configured to analyze the physiologic or functional signals received by the one or more ambulatory medical devices, and to confirm or reject one or more determinations made by one or more ambulatory medical devices, such as the implantable medical device 502, the wearable medical device 503, etc., or make additional determinations, etc. Computationally intensive algorithms, such as machine-learning algorithms, can be implemented in the external data processor.

With some examples, when parameter settings of an ambulatory medical device are analyzed using one or more trained machine learning models, and one or more differences between the parameter settings of the ambulatory medical device and the stored model parameter settings are detected, a recommendation to reprogram the medical device may be generated and presented to a clinician via a user interface of the remote device 508, or via a user interface of a software application executing on a client device communicatively connected with the remote device 508. The recommendation to reprogram the medical device may be determined by identifying differences between the parameter settings of the ambulatory medical device and the stored model parameter settings via the one or more machine learning models that otherwise went undetected by a clinician or a medical device programmer.

Portions of the one or more ambulatory medical devices or the external system 505 can be implemented using hardware, software, firmware, or combinations thereof. Portions of the one or more ambulatory medical devices or the external system 505 can be implemented using an application-specific circuit that can be constructed or configured to perform one or more functions or can be implemented using a general-purpose circuit that can be programmed or otherwise configured to perform one or more functions. Such a general-purpose circuit can include a microprocessor or a portion thereof, a microcontroller or a portion thereof, or a programmable logic circuit, a memory circuit, a network interface, and various components for interconnecting these components. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” can include electronic circuits configured to receive information and provide an electronic output representative of such received information.

A therapy device 510 can be configured to send information to or receive information from one or more of the ambulatory medical devices or the external system 505 using the communication link 511. In an example, the one or more ambulatory medical devices, the external device 506, or the remote device 508 can be configured to control one or more parameters of the therapy device 510. The external system 505 can allow for programming or reprogramming the one or more ambulatory medical devices and can receive information about one or more signals acquired by the one or more ambulatory medical devices, such as can be received via a communication link 511. The external system 505 can include a local external implantable medical device programmer. The external system 505 can include a remote patient management system that can monitor patient health status or adjust one or more therapies such as from a remote location.

In certain examples, event storage can be triggered, such as received physiologic information or in response to one or more detected events or determined parameters meeting or exceeding a threshold (e.g., a static threshold, a dynamic threshold, or one or more other thresholds based on patient or population information, etc.). Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected events can be stored, such as to increase the specificity of detection. In an example, multiple loop recorder windows (e.g., 2-minute windows, 4-minute windows, etc.) can be stored sequentially. In systems without early detection, to record this information, a loop recorder with a longer time period would be required at substantial additional cost (e.g., power, processing resources, component cost, amount of memory, etc.). Storing multiple windows using this early detection leading up to a single event can provide full event assessment with power and cost savings, in contrast to the longer loop recorder windows. In addition, the early detection can trigger additional parameter computation or storage, at different resolution or sampling frequency, without unduly taxing finite system resources.

In certain examples, one or more alerts can be provided, such as to the patient, to a clinician, or to one or more other caregivers (e.g., using a patient smart watch, a cellular or smart phone, a computer, etc.), in certain examples, in response to the transition to the high-power mode, in response to the detected event or condition, or after updating or transmitting information from a first device to a remote device. In other examples, the medical device itself can provide an audible or tactile alert to warn the patient of the detected condition. For example, the patient can be alerted in response to a detected condition so they can engage in corrective action, such as sitting down, etc.

In certain examples, a therapy can be provided in response to the detected condition. For example, a pacing therapy can be provided, enabled, or adjusted, such as to disrupt or reduce the impact of the detected event. In other examples, delivery of one or more drugs (e.g., a vasoconstrictor, pressor drugs, etc.) can be triggered, provided, or adjusted, such as using a drug pump, in response to the detected condition, alone or in combination with a pacing therapy, such as that described above, for example, to increase arterial pressure, to maintain cardiac output, to disrupt or reduce the impact of the detected event, or combinations thereof.

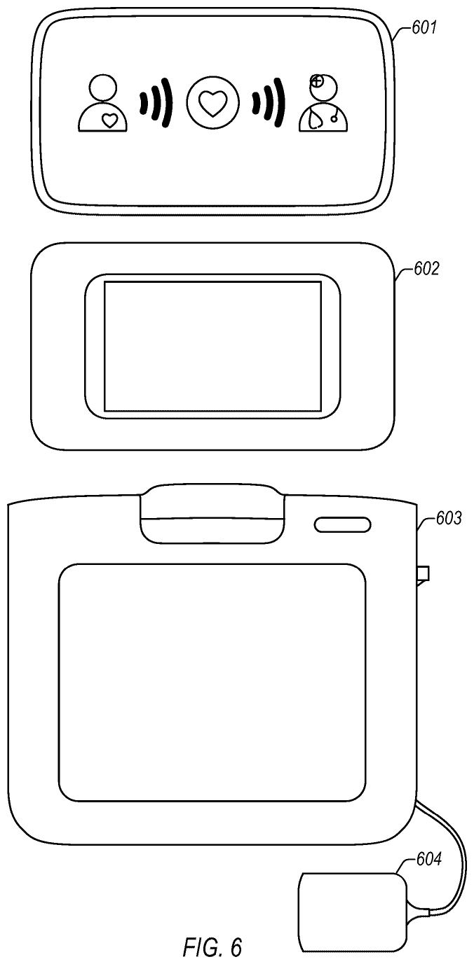

FIG. 6 illustrates example remote patient management systems for communication with an ambulatory medical device, such as for receiving information from or providing information to, including programming instructions, one or more ambulatory medical devices. The example remote patient management systems include a first remote patient management device 601 (e.g., a LATITUDE™ NXT Remote Patient Management System) for at-home monitoring and RF telemetry capabilities through one or more communication circuits with an ambulatory medical device and communication to a cloud-based server or clinician programming environment through a network connection, a second remote patient management device 602 (e.g., an EMBLEM™ S-ICD Programmer) with RF telemetry capabilities through one or more communication circuits and an optional external telemetry wand for communication with an ambulatory medical device, and a third remote patient management system 603 (e.g., a LATITUDE™ Programming System, Model 3300, etc.) with RF telemetry capabilities through one or more communication circuits and an external telemetry wand 604 (e.g., a Model 6395 Telemetry Wand, etc.) including an external telemetry coil configured for inductive communication with a corresponding telemetry coil of an implantable medical device. Although not illustrated herein, the remote patient management systems can include one or more other remote patient management systems, such as one or more other LATITUDE™ Programming systems, a remote patient monitoring application for a mobile device of a patient or other caregiver, etc. Each type of remote patient monitoring or management system has different capabilities and in certain examples permissions with respect to different programming instructions or features.

FIG. 7 illustrates a block diagram of an example machine 700 upon which any one or more of the techniques (e.g., methodologies) discussed herein may perform. Portions of this description may apply to the computing framework of one or more of the medical devices described herein, such as the implantable medical device, the external programmer, etc. Further, as described herein with respect to medical device components, systems, or machines, such may require regulatory-compliance not capable by generic computers, components, or machinery.

Examples, as described herein, may include, or may operate by, logic or a number of components, or mechanisms in the machine 700. Circuitry (e.g., processing circuitry, an assessment circuit, etc.) is a collection of circuits implemented in tangible entities of the machine 700 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine-readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 700 follow.

In alternative embodiments, the machine 700 may operate as a standalone device or may be connected (e.g., networked) to other machines. In a networked deployment, the machine 700 may operate in the capacity of a server machine, a client machine, or both in server-client network environments. In an example, the machine 700 may act as a peer machine in peer-to-peer (P2P) (or other distributed) network environment. The machine 700 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine 700 (e.g., computer system) may include a processor 702 (e.g., a central processing unit (CPU), a graphics processing unit (GPU), a hardware processor core, or any combination thereof), a main memory 704, a static memory 706 (e.g., memory or storage for firmware, microcode, a basic-input-output (BIOS), unified extensible firmware interface (UEFI), etc.), and mass storage 708 (e.g., hard drive, tape drive, flash storage, or other block devices) some or all of which may communicate with each other via an interlink 730 (e.g., a data bus). The machine 700 may further include a display unit 710, an input device 712 (e.g., a keyboard), and a user interface (UI) navigation device 714 (e.g., a mouse). In an example, the display unit 710, the input device 712, and the UI navigation device 714 may be a touch screen display. The machine 700 may additionally include a signal generation device 718 (e.g., a speaker), a network interface device 720, and one or more sensors 716, such as a global positioning system (GPS) sensor, compass, accelerometer, or one or more other sensors. The machine 700 may include an output controller 728, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 702, the main memory 704, the static memory 706, or the mass storage 708 may be, or include, a machine-readable medium 722 on which is stored one or more sets of data structures or one or more instructions 724 (e.g., software) embodying or utilized by any one or more of the techniques or functions described herein. The one or more instructions 724 may also reside, completely or at least partially, within any of registers of the processor 702, the main memory 704, the static memory 706, or the mass storage 708 during execution thereof by the machine 700. In an example, one or any combination of the processor 702, the main memory 704, the static memory 706, or the mass storage 708 may constitute the machine-readable medium 722. While the machine-readable medium 722 is illustrated as a single medium, the term “machine-readable medium” may include a single medium or multiple media (e.g., a centralized or distributed database, and/or associated caches and servers) configured to store the one or more instructions 724.

The term “machine-readable medium” may include any medium that is capable of storing, encoding, or carrying instructions for execution by the machine 700 and that cause the machine 700 to perform any one or more of the techniques of the present disclosure, or that is capable of storing, encoding, or carrying data structures used by or associated with such instructions. Non-limiting machine-readable medium examples may include solid-state memories, optical media, magnetic media, and signals (e.g., radio frequency signals, other photon-based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine-readable media that do not include transitory propagating signals. Specific examples of non-transitory machine-readable media may include non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The one or more instructions 724 may be further transmitted or received over a communications network 726 using a transmission medium via the network interface device 720 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 720 may include one or more physical jacks (e.g., Ethernet, coaxial, or phone jacks) or one or more antennas to connect to the communications network 726. In an example, the network interface device 720 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 700, and includes digital or analog communications signals or other intangible medium to facilitate communication of such software. A transmission medium is a machine-readable medium.

Various embodiments are illustrated in the figures above. One or more features from one or more of these embodiments may be combined to form other embodiments. Method examples described herein can be machine or computer-implemented at least in part. Some examples may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A medical device system to improve resource utilization in ambulatory patient monitoring, comprising:
an ambulatory medical device configured to perform long-term ambulatory monitoring of physiologic information of a patient, the ambulatory medical device comprising:
a communication circuit configured to communicate with a remote device, including to receive a request to record a patient-triggered episode; and
a control circuit configured to control operation of the ambulatory medical device, including, in response to receiving the request to record the patient-triggered episode, to transition the ambulatory medical device from a long-term ambulatory monitoring state to a patient-triggered episode state,
wherein the control circuit, in the patient-triggered episode state, is configured to:
control recording and storage of a set of physiologic information of the patient for the patient-triggered episode; and
control, at a first time, communication of a first subset of physiologic information to the remote device and storage of a second subset of physiologic information at the ambulatory medical device for selectable separate and subsequent communication to the remote device to improve resource utilization of the ambulatory patient monitoring.

2. The medical device system of claim 1, wherein the control circuit, in the patient-triggered episode state, is configured to partition the set of physiologic information of the patient for the patient-triggered episode into the first subset of physiologic information and the second subset of physiologic information,
wherein the first subset of physiologic information has a size or duration smaller than a size or duration of the set of physiologic information for the patient-triggered episode,
wherein, at the first time, the control circuit is configured to control communication of the first subset of physiologic information to the remote device and storage of the second subset of physiologic information at the ambulatory medical device without communicating the second subset of physiologic information to the remote device at the first time to improve resource utilization of the ambulatory patient monitoring.

3. The medical device system of claim 1, wherein the ambulatory medical device comprises a cardiac electrical sensor configured to sense cardiac electrical information of the patient,
wherein, in the long-term ambulatory monitoring state, the cardiac electrical sensor is configured to detect a heart rate or cardiac interval of the patient,
wherein, in the patient-triggered episode state, the cardiac electrical sensor is configured to capture a subcutaneous electrocardiogram of the patient having a higher resolution or sampling frequency than the cardiac electrical information detected in the long-term ambulatory monitoring state,
wherein the set of physiologic information of the patient for the patient-triggered episode comprises the subcutaneous electrocardiogram.

4. The medical device system of claim 1, wherein the ambulatory medical device comprises an implantable medical device,
wherein the medical device system comprises the remote device and the remote device is configured to:
receive a request from the patient to record the patient-triggered episode and provide the request to record the patient-triggered episode to the ambulatory medical device;
receive, in response to the request to record the patient-triggered episode, the first subset of physiologic information from the ambulatory medical device;
analyze the first subset of physiologic information using one or more algorithms to detect a cardiac rhythm of interest in the first subset of physiologic information; and
generate, in response to detecting the cardiac rhythm of interest in the first subset of physiologic information, an alert for clinical review of the patient-triggered episode including an indication that additional physiologic information of the patient-triggered episode is available for transmission at the ambulatory medical device.

5. The medical device system of claim 4, wherein to analyze the first subset of physiologic information to detect the cardiac rhythm of interest includes to detect at least one of:
atrioventricular block;
atrial fibrillation;
one or more or a series of premature ventricular contractions;
a supraventricular tachycardia;
one or more ectopic beats;
sinus bradycardia;
sinus tachycardia; or
junctional rhythms.

6. The medical device system of claim 4, wherein the remote device is configured to:
request, in response to detecting the cardiac rhythm of interest in the first subset of physiologic information, the second subset of physiologic information from the ambulatory medical device;
receive, in response to the request for the second subset of physiologic information, the second subset of physiologic information from the ambulatory medical device; and
analyze the second subset of physiologic information to detect or confirm the cardiac rhythm of interest in the patient-triggered episode or the second subset of physiologic information, annotate the patient-triggered episode with a label to identify the detected cardiac rhythm of interest, and generate an alert for clinical review of the annotated patient-triggered episode including an indication that additional physiologic information of the patient is available for transmission at the ambulatory medical device.

7. The medical device system of claim 4, comprising a remote server separate from the remote device, wherein the remote server is configured, in response to the remote device detecting the cardiac rhythm of interest in the first subset of physiologic information, to:
receive the first and second subsets of physiologic information from the ambulatory medical device or the remote device;
analyze the first and second subsets of physiologic information using a deep learning cardiac algorithm to detect a cardiac arrhythmia in the physiologic information of the patient-triggered episode;
annotate the patient-triggered episode with a label to identify the detected cardiac rhythm of interest;
generate a programming change for the ambulatory medical device based on the detected cardiac arrhythmia; and
generate an alert for clinical review of the annotated patient-triggered episode and the generated programming change.

8. The medical device system of claim 7, wherein the remote server is configured to prioritize the patient-triggered episode for clinical review based on the detected cardiac rhythm of interest, including to flag critical arrhythmias for urgent clinical review.

9. The medical device system of claim 1, wherein the communication circuit is configured to receive a request to transmit the second subset of physiologic information to the remote device,
wherein the control circuit, in response to receiving the request to transmit the second subset of physiologic information to the remote device, is configured to control, at a second time separate from and subsequent to the first time, communication of the second subset of physiologic information to the remote device or a remote server separate from the remote device.

10. The medical device system of claim 9, wherein, in response to receiving the request to transmit the second subset of physiologic information to the remote device, the control circuit is configured to enable monitoring of additional physiological information of the patient using one or more sensors of the ambulatory medical device and communication of the additional physiologic information to the remote device or the remote server separate from the remote device, the additional physiologic information comprising at least one of:
blood pressure information using a blood pressure sensor;
temperature information using a temperature sensor;
oxygen saturation information using an oxygen saturation sensor;
photographic information of the patient using an image sensor;
heart sound information using a heart sound sensor;
activity information using an activity sensor; or
impedance information using an impedance sensor.

11. A method to improve resource utilization in ambulatory patient monitoring, comprising:
receiving, using a communication circuit of an ambulatory medical device, a request to record a set of physiologic information of a patient for a patient-triggered episode using one or more sensors of the ambulatory medical device;
transitioning, in response to receiving the request to record the patient-triggered episode, the ambulatory medical device from a long-term ambulatory monitoring state to a patient-triggered episode state;
controlling, in the patient-triggered episode state, recording and storage of the set of physiologic information of the patient for the patient-triggered episode; and
controlling, at a first time, in the patient-triggered episode state, communication of a first subset of physiologic information to a remote device and storage of a second subset of physiologic information at the ambulatory medical device for selectable separate and subsequent communication to the remote device to improve resource utilization of the ambulatory patient monitoring.

12. The method of claim 9, comprising:
partitioning the set of physiologic information of the patient for the patient-triggered episode into the first subset of physiologic information and the second subset of physiologic information,
wherein the first subset of physiologic information has a size or duration smaller than a size or duration of the set of physiologic information for the patient-triggered episode,
wherein controlling communication of the first subset of physiologic information to the remote device and storage of the second subset of physiologic information at the ambulatory medical device at the first time comprises without communicating the second subset of physiologic information to the remote device at the first time to improve resource utilization of the ambulatory patient monitoring.

13. The method of claim 9, comprising sensing cardiac electrical information of the patient using a cardiac electrical sensor, including:
in the long-term ambulatory monitoring state, detecting a heart rate or cardiac interval of the patient using the cardiac electrical sensor; and
in the patient-triggered episode state, capturing a subcutaneous electrocardiogram of the patient using the cardiac electrical sensor, the subcutaneous electrocardiogram having a higher resolution or sampling frequency than the cardiac electrical information detected in the long-term ambulatory monitoring state,
wherein the set of physiologic information of the patient for the patient-triggered episode comprises the subcutaneous electrocardiogram.

14. The method of claim 9, comprising, using the remote device:
receiving a request from the patient to record the patient-triggered episode and provide the request to record the patient-triggered episode to the ambulatory medical device;
receiving, in response to the request to record the patient-triggered episode, the first subset of physiologic information from the ambulatory medical device;
analyzing the first subset of physiologic information using one or more algorithms to detect a cardiac rhythm of interest in the first subset of physiologic information; and
generating, in response to detecting the cardiac rhythm of interest in the first subset of physiologic information, an alert for clinical review of the patient-triggered episode including an indication that additional physiologic information of the patient-triggered episode is available for transmission at the ambulatory medical device.

15. The method of claim 14, wherein analyzing the first subset of physiologic information to detect the cardiac rhythm of interest includes detecting at least one of:
atrioventricular block;
atrial fibrillation;
one or more or a series of premature ventricular contractions;
a supraventricular tachycardia;
one or more ectopic beats;
sinus bradycardia;
sinus tachycardia; or
junctional rhythms.

16. The method of claim 14, comprising, using the remote device:
receiving, in response to detecting the cardiac rhythm of interest in the first subset of physiologic information, the second subset of physiologic information from the ambulatory medical device; and
analyzing the second subset of physiologic information to detect or confirm the cardiac rhythm of interest in the patient-triggered episode or the second subset of physiologic information, annotate the patient-triggered episode with a label to identify the detected cardiac rhythm of interest, and generate an alert for clinical review of the annotated patient-triggered episode including an indication that additional physiologic information of the patient is available for transmission at the ambulatory medical device.

17. The method of claim 14, comprising, using a remote server separate from the remote device, in response to the remote device detecting the cardiac rhythm of interest in the first subset of physiologic information:
receiving the first and second subsets of physiologic information from the ambulatory medical device or the remote device;
analyzing the first and second subsets of physiologic information using a deep learning cardiac algorithm to detect a cardiac arrhythmia in the physiologic information of the patient-triggered episode;
annotating the patient-triggered episode with a label to identify the detected cardiac rhythm of interest;
generating a programming change for the ambulatory medical device based on the detected cardiac arrhythmia; and
generating an alert for clinical review of the annotated patient-triggered episode and the generated programming change.

18. The method of claim 17, comprising, using the remote server:
prioritizing the patient-triggered episode for clinical review based on the detected cardiac rhythm of interest, including to flag critical arrhythmias for urgent clinical review.

19. The method of claim 11, comprising:
receiving, using the communication circuit, a request to transmit the second subset of physiologic information to the remote device; and
controlling, using the control circuit, in response to receiving the request to transmit the second subset of physiologic information to the remote device, at a second time separate from and subsequent to the first time, communication of the second subset of physiologic information to the remote device or a remote server separate from the remote device.

20. The method of claim 19, comprising:
enabling, using the control circuit and in response to receiving the request to transmit the second subset of physiologic information to the remote device, monitoring of additional physiological information of the patient using the one or more sensors and communication of the additional physiologic information to the remote device or the remote server separate from the remote device, the additional physiologic information comprising at least one of:
blood pressure information using a blood pressure sensor;
temperature information using a temperature sensor;
oxygen saturation information using an oxygen saturation sensor;
photographic information of the patient using an image sensor;
heart sound information using a heart sound sensor;
activity information using an activity sensor; or
impedance information using an impedance sensor.
